(19) United States
(12) Patent Application Publication
(10) Pub. No.: US 2011/0059861 A1
(43) Pub. Date: Mar. 10, 2011

(54) ANALYSIS OF CELL NETWORKS

(75) Inventors: Garry P. Nolan, San Francisco, CA (US); Alessandra Cesano, Redwood City, CA (US)

(73) Assignee: Nodality, Inc., South San Francisco, CA (US)

(21) Appl. No.: 12/877,998

(22) Filed: Sep. 8, 2010

Related U.S. Application Data
(60) Provisional application No. 61/240,613, filed on Sep. 8, 2009.

(51) Int. Cl. C12Q 1/68; C12Q 1/02; C40B 30/06; G01N 27/26; G01N 33/53; G06F 19/00; G06F 17/10

(52) U.S. Cl. 506/10; 435/29; 435/6; 204/456; 435/7.92; 702/19; 703/2

(57) ABSTRACT

The present invention provides an approach for the determination of activation state of a plurality of discrete cell populations and/or the state of one or more cellular networks in an individual. The status of a plurality of discrete cell populations and/or the state of one or more cellular networks can be correlated with the diagnosis, prognosis, choice or modification of treatment, and/or monitoring of a condition

### CROSS-REFERENCE

This application claims the benefit of U.S. Provisional Application No. 61/240,613 filed Sep. 8, 2009, which application is incorporated herein by reference.

### BACKGROUND OF THE INVENTION

Many conditions are characterized by disruptions in cellular pathways that lead, for example, to aberrant control of cellular processes, with uncontrolled growth and increased cell survival. These disruptions are often caused by changes in the activity of molecules participating in cellular pathways. For example, alterations in specific signaling pathways have been described for many cancers.

Conditions today are diagnosed by analyzing these disruptions in a single homogenous population of cells. However, different types of cells co-exist with other different types of cells in a complex environment milieu which might affect the pathology of a condition. Thus, the successful diagnosis of a condition and use of therapies may require knowledge of the cellular events that are responsible for the condition pathology in a variety of cells and cellular networks.

Accordingly, there is a need for a biologically based clinically relevant analysis of condition disorders that can predict the disease course for an individual. This analysis, based upon the status of different discrete cell populations and/or environmental inputs will provide a complete depiction of the pathology of a condition, thus, aiding clinicians in both more reliable prognosis and therapeutic selection at the individual patient level.

### SUMMARY OF THE INVENTION

In some embodiments, the invention is directed to methods of determining the status of an individual, by: a) contacting a first cell from a first cell population from the individual with at least a first modulator; b) contacting a second cell from a second cell population from the individual with at least a second modulator; c) determining an activation level of at least one activatable element in the first cell and the second cell; d) creating a response panel for the individual comprising the determined activation levels of the activatable elements; and e) identifying the status of the individual, where the identifying is based on the response panel. In some embodiments, the invention further comprises determining a causal between the first cell and the second cell based on the response panel, where the causal association is indicative of a state of a cell network. In some embodiments, the invention further comprises applying a classifier to a response panel and/or a state cell network; where the classifier comprises a set of activation levels values, and where the classifier is used to determine whether the response panel and/or cell network is associated with the status of the individual. In some embodiments, the methods of the invention further comprise generating a classification value based on the response panel, where the classification value specifies whether the individual is associated with a status of the individual. In some embodiments, the status of the individual is a classification, diagnosis, or prognosis of a condition. In some embodiments, the AUC value in the classification, diagnosis, or prognosis of the condition is higher than 0.6. In some embodiments, the p value in the classification, diagnosis, or prognosis of the condition is below 0.05. In some embodiments, the positive predictive value (PPV) in the classification, diagnosis, or prognosis of the condition is higher than 80%. In some embodiments, the negative predictive value (NPV) in the classification, diagnosis, or prognosis of the condition is higher than 80%.

In some embodiments, the first and second modulator are selected from the group consisting of growth factor, mitogen, cytokine, chemokine, adhesion molecule modulator, hormone, small molecule, polynucleotide, antibody, natural compound, lactone, chemotherapeutic agent, immune modulator, carbohydrate, protease, ion, reactive oxygen species, and radiation. In some embodiments, the first modulator and second modulator are the same. In some embodiments, the contacting of the first cell and the second cell is in a same culture. In some embodiments, the first modulator and second modulator are different. In some embodiments, the contacting of the first cell and the second cell are in separate cultures. In some embodiments, the contacting of the first cell and/or the contacting of the second cell is before isolation of the first cell and/or the second cell from the individual.

In some embodiments, the activation level is based on an activation state selected from the group consisting of extracellular protease exposure, novel hetero-oligomer formation, glycosylation state, phosphorylation state, acetylation state, methylation state, biotinylation state, glutamylation state, glycylation state, hydroxylation state, isomerization state, prenylation state, myristoylation state, lipoylation state, phosphopantetheinylation state, sulfation state, ISGylation state, nitrosylation state, palmitoylation state, SUMOylation state, ubiquitination state, neddylation state, citrullination state, deamidation state, disulfide bond formation state, proteolytic cleavage state, translocation state, changes in protein turnover, multi-protein complex state, oxidation state, multi-lipid complex, and biochemical changes in cell membrane. In some embodiments, the activation state is a phosphorylation state.

In some embodiments, the activatable element is selected from the group consisting of proteins, carbohydrates, lipids, nucleic acids and metabolites. In some embodiments, the activatable element is a protein capable of being to phosphoryled and/or dephosphorylated.

In some embodiments, the method further comprises determining the presence or absence of one or more cell surface markers, intracellular markers, or combination thereof in the first cell and/or the second cell. In some embodiments, the cell surface markers and the intracellular markers are independently selected from the group consisting of proteins, carbohydrates, lipids, nucleic acids and metabolites. In some embodiments, determining of the presence or absence of one or more cell surface markers or intracellular markers comprises determining the presence or absence of an epitope in both activated and non-activated forms of the cell surface markers or the intracellular markers. In some embodiments, the status of the individual is based on both the activation levels of the activatable elements and the presence or absence of the one or more cell surface markers, intracellular markers, or combination thereof.

In some embodiments, the activation level is determined by a process comprising the binding of a binding element which is specific to a particular activation state of the particular activatable element. In some embodiments, the binding element comprises an antibody. In some embodiments, the binding elements are distinguishably labeled. In some embodiments, the distinguishably labeled binding element is directly labeled with a detectable label. In some embodiments, the detectable label is selected from the group consisting of radioisotopes, heavy isotopes, fluorescers, FRET labels, enzymes, particles, and chemiluminescers.

In some embodiments, the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells. In some embodiments, the step of determining the activation level comprises the use of flow cytometry. In some embodiments, the determining step is quantitative. In some embodiments, the determining step is relative to a control value. In some embodiments, the control value is included in the response panel.

In some embodiments, the status of the individual is the classification, diagnosis, prognosis of a condition. In some embodiments, the condition is an immunologic, malignant, or proliferative disorder or a combination thereof. In some embodiments the condition is a malignant disorder. In some embodiments, the malignant disorder is a solid tumor or a hematologic malignancy.

In some embodiments, the malignant disorder is non-B cell lineage derived. In some embodiments, the non-B cell lineage derived malignant disorder is selected from the group consisting of Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B cell atypical immune lymphoproliferations. In some embodiments, the non-B cell lineage derived malignant disorder is AML.

In some embodiments, the malignant disorder is a B cell or B cell lineage derived disorder. In some embodiments, the malignant disorder is a B-Cell or B cell lineage derived disorder selected from the group consisting of Chronic Lymphocytic Leukemia (CLL), B cell lymphocyte lineage leukemia, B cell lymphocyte lineage lymphoma, Multiple Myeloma, and plasma cell disorders. In some embodiments, the B-Cell or B cell lineage derived disorder is CLL.

In some embodiments, the status of the individual is a predicted response to a treatment for a pre-pathological or pathological condition, or a response to treatment for a pre-pathological or pathological condition. In some embodiments, the methods further comprise predicting a response to a treatment for a pre-pathological or pathological condition.

In some embodiments, the activation levels of a plurality of intracellular activatable elements in the first cell and/or second cell is determined

In some embodiments, the invention provides a computer-implemented method of classifying activation state data derived from a population of cells according to a characteristic, the method comprising: providing a computer comprising memory and a processor; identifying an activation state data associated with an individual, where the activation state data is derived from at least two discrete populations of cells sampled from an individual; generating a classification value, where the classification value specifies whether the individual is associated with a health status responsive to applying a classifier to the activation state data associated with the individual; where the classifier comprises a set of activation state values used to determine whether cells in different discrete populations of cells are associated with the status; and storing the classification value in memory associated with the computer. In some embodiments, the classification value represents one or more of the following: a diagnosis, a prognosis and a predicted response to treatment.

In some embodiments, the activation state data is received from a third party and further comprising: transmitting the classification value to the third party. In some embodiments, the methods further comprise: identifying whether the activation state data is associated with a first discrete population of cells or a second distinct population of cells based on at least a first level of an activation state associated with an activatable element. In some embodiments, identifying whether the activation state data is associated with the first discrete population of cells or the second distinct population of cells comprises gating the activation state data based on the at least a first level of an activation state associated with the activatable element. In some embodiments, identifying whether the activation state data is associated with the first discrete population of cells or the second discrete population of cells comprises iteratively binning the activation state data based on at least a first level of an activation state associated with an activatable element.

In some embodiments, the first discrete population of cells is a rare population of cells and the first discrete population of cells is identified responsive to iteratively binning the activation state data based on at least a first level of an activation state associated with an activatable element.

In some embodiments, the methods further comprise generating the classifier based on activation state data derived from a plurality of discrete populations of cells that are known to be associated with the status and a plurality of discrete populations of cells that are know not to be associated with the status. In some embodiments, the activation state data is further associated with a plurality of time points and generating the classifier further comprises: generating a model of the data over the different time points, where the model represents communications between the heterogeneous populations of cells over the plurality of time points; generating a series of descriptive values based on the model; and generating the classifier based on the series of descriptive values. In some embodiments, generating the classifier comprises cross-validating the classifier

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an example of the immune system cell communication network.

FIG. 2 illustrates different activation levels of pStat1, pStat3 and pStat5 in lymphocytes, nRBC1 cells, Myeloid(p1) cells and stem cells after treatment with EPO, G-CSF and EPO+G-CSF.

FIG. 3 illustrates a kinetic responses of different discrete cell populations in normal samples.

### DETAILED DESCRIPTION OF THE INVENTION

The present invention incorporates information disclosed in other applications and texts. The following patent and other publications are hereby incorporated by reference in their entireties: Haskell et al, Cancer Treatment, 5th Ed., W.B. Saunders and Co., 2001; Alberts et al., The Cell, 4th Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7th Ed., Garland, and Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.; and Sambrook, Fritsche and Maniatis. “Molecular Cloning A laboratory Manual” 3rd Ed. Cold Spring Harbor Press (2001), all of which are herein incorporated in their entirety by reference for all purposes.

One embodiment of the invention is directed to methods for determining the status of an individual by determining the activation level of cells in different discrete populations of cells obtained from the individual. Typically, the status of an individual will be a status related to the health of the individual (referred to herein as “health status” or “disease status”), but any type of status can be determined if it can be correlated to the status of cells (e.g. single cells) from one or more discrete populations of cells from the individual. In some embodiments, the invention provides methods for determining the status of an individual by creating a response panel using two or more discrete cell populations. In some embodiments, the status of an individual is determined by a method comprising: a) contacting a first cell from a first discrete cell population from said individual with at least a first modulator; b) contacting a second cell from a second discrete cell population from said individual with at least a second modulator; d) determining an activation level of at least one activatable element in said first cell and said second cell; e) creating a response panel for said individual comprising said determined activation levels of said activatable elements; and f) making a decision regarding the status of said individual, wherein said decision is based on said response panel. Thus, the invention provides methods for the determination of the status of an individual by analyzing a plurality (e.g. two or more) of discrete populations of cells. In some embodiments, the invention provides a method to demarcate discrete populations of cells that correlate with a clinical outcome for a disease. In some embodiments, the invention provides different discrete populations of cells which analysis in combination allows for the determination of the status of an individual. In some embodiments, the invention provides different discrete populations of cells which analysis in combination allows for the determination of the state of a cellular network. In some embodiments, the invention provides for the determination of a causal association between discrete populations of cells, where the causal association is indicative of the status of a cell network. In another embodiment, the invention provides a method to determine whether one or more cell populations that are part of a cellular network are associated with a status.

The status of an individual may be associated with a diagnosis, prognosis, choice or modification of treatment, and/or monitoring of a disease, disorder, or condition. Through the determination of the status of an individual, a health care practitioner can assess whether the individual is in the normal range for a particular condition or whether the individual has a pre-pathological or pathological condition warranting monitoring and/or treatment. Thus, in some embodiments, the status of an individual involves the classification, diagnosis, prognosis of a condition or outcome after administering a therapeutic to treat the condition.

One embodiment of the present invention involves the classification, diagnosis, prognosis of a condition or outcome after administering a therapeutic to treat the condition. Another embodiment of the invention involves monitoring and predicting outcome of a condition. Another embodiment is drug screening using some of the methods of the invention, to determine which drugs may be useful in particular conditions. In some embodiments, an analysis method involves evaluating cell signals and/or expression markers in different discrete cell populations in performing these processes. One embodiment of cell signal analysis involves the analysis of one or more phosphorylated proteins (e.g. by flow cytometry) in different discrete cell populations. The classification, diagnosis, prognosis of a condition and/or outcome after administering a therapeutic to treat the condition is then determined based in the analysis of the one or more phosphorylated proteins in different discrete cell populations. In one embodiment, a signal transduction-based classification of a condition can be performed using clustering of phospho-protein patterns or biosignatures of the different cell discrete populations.

In some embodiments, a treatment is chosen based on the characterization of a plurality of discrete cell populations. In some embodiments, characterizing a plurality of discrete cell populations comprises determining the activation state of one or more activatable elements in the plurality of cell populations. The activatable element(s) analyzed among the plurality of discrete cell populations can be the same or can be different.

In some embodiments, the present invention provides methods for classification, diagnosis, prognosis of a condition or outcome after administering a therapeutic to treat the condition by characterizing one or more pathways in different discrete cell populations. In some embodiments, a treatment is chosen based on the characterization of the pathway(s) simultaneously in the different discrete cell populations. In some embodiments, characterizing one or more pathways in different discrete cell populations comprises determining whether apoptosis pathways, cell cycle pathways, signaling pathways, or DNA damage pathways are functional in the different discrete cell populations based on the activation levels of one or more activatable elements within the pathways, where a pathway is functional if it is permissive for a response to a treatment.

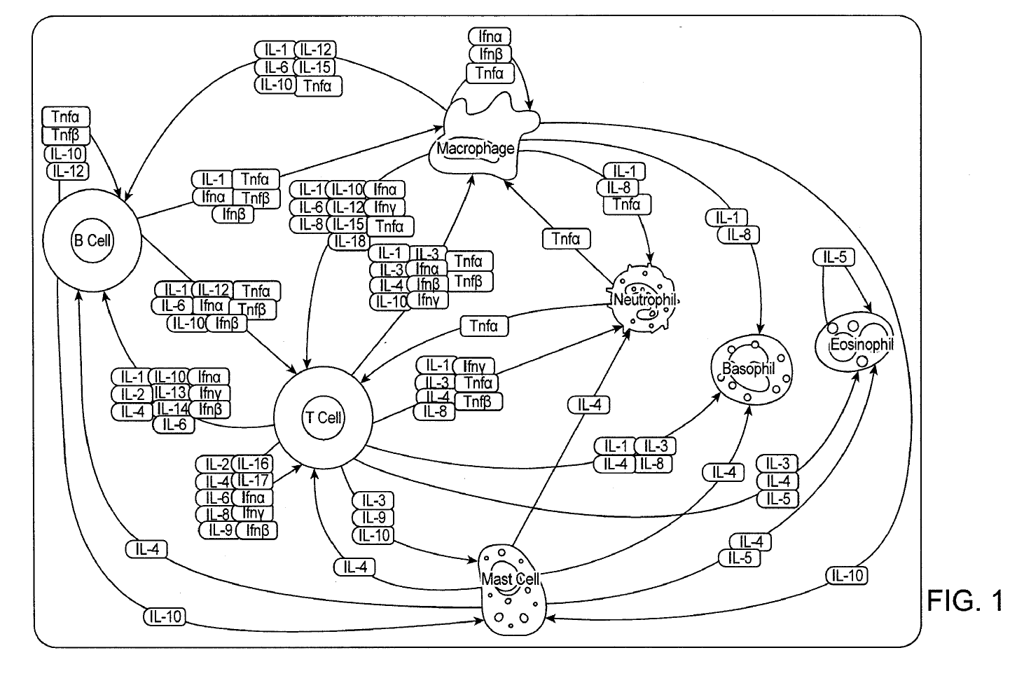

In some embodiments, the characterization of different discrete cell populations in a condition (e.g. cancer) shows disruptions in cellular networks that are reflective of increased proliferation, increased survival, evasion of apoptosis, insensitivity to anti-growth signals and other mechanisms. In some embodiments, the disruption in these networks can be revealed by exposing a plurality of discrete cell populations to one or more modulators that mimic one or more environmental cues. FIG. 1 shows an example of how the biology of a plurality of discrete cell populations in the immune system can determine the pathology of a condition and outcome. For example, without intending to be limited to any theory, several different cell types participate as part of the immune system, including B cells, T cells, macrophages, neutrophils, basophils and eosinophils. Each of these cell types has a distinct role in the immune system, and communicates with other immune cells using secreted factors called cytokines, including interleukins, TNF, and the interferons. Macrophages phagocytose foreign bodies and are antigen-presenting cells, using cytokines to stimulate specific antigen dependent responses by B and T cells and non-specific responses by other cell types. T cells secrete a variety of factors to coordinate and stimulate immune responses to specific antigen, such as the role of helper T cells in B cell activation in response to antigen. The proliferation and activation of eosinophils, neutrophils and basophils respond to cytokines as well. Cytokine communication is often local, within a tissue or between cells in close proximity. Each of the cytokines is secreted by one set of cells and provokes a response in another target set of cells, often including the cell that secretes the cytokine.

In response to tissue injury, a multifactorial network of chemical signals initiate and maintain a host response designed to heal the afflicted tissue. When a condition such as cancer is present in an individual the homeostasis in, e.g., tissue, organ and/or microenvironment is perturbed. For example, neoplasia-associated angiogenesis and lymphangiogenesis produces a chaotic vascular organization of blood vessels and lymphatics where neoplastic cells interact with other cell types (mesenchymal, haematopoietic and lymphoid) and a remodelled extracellular matrix. Neoplastic cells produce an array of cytokines and chemokines that are mitogenic and/or chemoattractants for granulocytes, mast cells, monocytes/macrophages, fibroblasts and endothelial cells. In addition, activated fibroblasts and infiltrating inflammatory cells secrete proteolytic enzymes, cytokines and chemokines, which are mitogenic for neoplastic cells, as well as endothelial cells involved in neoangiogenesis and lymphangiogenesis. These factors can potentiate tumor growth, stimulate angiogenesis, induce fibroblast migration and maturation, and enable metastatic spread via engagement with either the venous or lymphatic networks. Thus, determining the activation state data of various cell populations in an individual provides a better picture of the status of the individual and/or the state of the cellular network.

In a condition like rheumatoid arthritis (RA) contributions made by interactions between dendritic cells, T cells and other immune cells, and local production of cytokines and chemokines may contribute to the pathogenesis of RA. These cells further interact with local cells (e.g. synoviocytes). In response to local inflammation and production of proinflammatory cytokines, after unknown event dendritic cells, T cells and other immune cells are attracted to the synovium in response to local production of cytokines and chemokines. In some patients with rheumatoid arthritis, chronic inflammation leads to the destruction of the cartilage, bone, and ligaments, causing deformity of the joints. Damage to the joints can occur early in the disease and be progressive.

The determination of the status (e.g. health status, disease status and/or any status indicating the pathophysiology of an individual) may also indicate response of an individual to treatment for a condition. Such information allows for ongoing monitoring of the condition and/or additional treatment. In one embodiment, the invention provides for the detection of the presence of disease-associated cells or the absence or reduction of cells necessary for normal physiology in an individual that is being treated, or was previously treated, for the disease or condition. In some embodiments, the status may also indicate predicted response to a treatment.

In some embodiments, the determination of the status of an individual may be used to ascertain whether a previous condition or treatment has induced a new pre-pathological or pathological condition that requires monitoring and/or treatment. For example, treatment for many forms of cancers (e.g. lymphomas and childhood leukemias) can induce certain adult leukemias, and the methods of the present invention allow for the early detection and treatment of such leukemias.

In a further embodiment, the status of an individual may indicate an individual's immunologic status and may reflect a general immunologic status, an organ or tissue specific status, or a disease related status.

The subject invention also provides kits (described in detail below in the section entitled “Kits”) for use in determining the status of an individual, the kit comprising one or more specific binding elements for signaling molecules, and may additionally comprise one or more therapeutic agents. The kit may further comprise a software package for data analysis of the different populations of cells, which may include reference profiles for comparison with the test profile.

The discussion below describes some of the preferred embodiments with respect to particular diseases. However, it should be appreciated that the principles may be useful for the analysis of many other diseases as well.

### INTRODUCTION

Cells respond to environmental and systemic signals to adjust their responses to varying demands. For example, cells respond to factors such as hormones, growth factors and cytokine produced by other cells or from the environment. Cells also respond to injury and physiological changes. As a result, each tissue, organ, microenvironment (e.g. niche) or cell has the capacity to modulate the activity of cells. In addition, the presence of cells (e.g. cancer cells) can have influence in a surrounding tissue, organ, microenvironment (e.g. niche) or cell.

A cell might be passive in the communication with a surrounding tissue, organ, microenvironment (e.g. niche) or cell, merely adjusting their activity levels according to the environment demands A cell might influence a surrounding tissue, organ, microenvironment (e.g. niche) or cell by virtue of progeny or signals such as cell contacts, secreted or membrane bounds factors. Thus, cells co-exist with other types of cells in a complex environment milieu. Different types of cells that interact with each other in a tissue, an organ, or a microenvironment such as a niche participate in a network that might determine the status of an individual (e.g. developing of a condition or performing normal functions).

A discrete cell population, as used herein, refers to a population of cells in which the majority of cells is of a same cell type or has a same characteristic. For many years, research into several conditions (e.g. cancer) has focused on attempts to identify a causative cell population comprised of cells of a single cell type. However, several discrete cell populations or the interactions between several cell populations may contribute to the pathology of a condition. For example, in the case of a cancer cell, the cancer cell may possess a dysregulated response to an environmental cue (e.g. cytokine) such that the cell proliferates rather than undergo apoptosis. Alternatively, the environment in which the cell is located (e.g. niche, tissue, organ) may abnormally produce a factor that causes the cancer cell to undergo uncontrolled proliferation. In addition, the cancer cell may produce one or more factors that influence its environment (e.g. niche, tissue, organ), and, as a result the pathology of the cancer is worsened.

Thus, the successful diagnosis of a condition and use of therapies may require knowledge of the activation state data of different discrete cell populations that may play a role in the pathogenesis of a condition (e.g. cancer). The determination of the activation state data of different discrete cell populations that might interact directly or indirectly in a network serves as an indicator of the state of the network. In addition, it provides directionality to the interactions among the different discrete cell populations in the network. It also provides information across the cell populations participating in the network. As a result, the determination of activation state data of different discrete cell populations may serve as a better indicator of a condition than the analysis of a single discrete cell population.

In some embodiments, the activation state data of a plurality of populations of cells is determined by analyzing multiple single cells in each population (e.g. by flow cytometry). Measuring multiple single cells in each discrete cell population in an individual provides multiple data points that in turn allows for the determination of the network boundaries in the individual. Measuring modulated networks at a single cell level provides the lever of biologic resolution that allows the assessment of intrapatient clonal heterogeneity ultimately relevant to disease management and outcome. The network boundaries and/or the state of the network might change when the individual is suffering from a pathological condition or if the individual is responding or not responding to treatment. Thus, the determination of network boundaries and/or the state of the network can be used for diagnosis, prognosis of a condition or determination of outcome after administering a therapeutic to treat the condition.

One aspect of the invention provides methods for determining the status of an individual by analyzing different discrete cell populations in said individual. In some embodiments, the invention provides methods for determining the state of a cellular network. The cellular network can be correlated with the status of an individual. In some embodiments, determining the status of an individual involves the classification, diagnosis, prognosis of a condition or outcome after administering a therapeutic to treat the condition.

### Samples and Sampling

The methods involve analysis of one or more samples from an individual. An individual or a patient is any multi-cellular organism; in some embodiments, the individual is an animal, e.g., a mammal. In some embodiments, the individual is a human.

The sample may be any suitable type that allows for the analysis of different discrete populations of cells. The sample may be any suitable type that allows for the analysis of single populations cells. Samples may be obtained once or multiple times from an individual. Multiple samples may be obtained from different locations in the individual (e.g., blood samples, bone marrow samples and/or lymph node samples), at different times from the individual (e.g., a series of samples taken to monitor response to treatment or to monitor for return of a pathological condition), or any combination thereof. These and other possible sampling combinations based on the sample type, location and time of sampling allows for the detection of the presence of pre-pathological or pathological cells, the measurement treatment response and also the monitoring for disease.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cancer may be sampled (e.g., by blood draw) relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic lavage), vagina, bladder (bladder irrigation), breast duct (ductal lavage), oral, nasal, sinus cavities, and peritoneal cavity (peritoneal cavity perfusion). In some embodiments the sample or samples is blood.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

Samples may include circulating tumor cells (CTC). Methods for isolating CTC are known in the art. See for example: Toner M et al. Nature 450, 1235-1239 (20 Dec. 2007); Lustenberger P et al. Int J. Cancer. 1997 Oct. 21; 74(5):540-4; Reviews in Clinical Laboratory Sciences, Volume 42, Issue 2 Mar. 2005, pages 155-196; and Biotechno, pp. 109-113, 2008 International Conference on Biocomputation, Bioinformatics, and Biomedical Technologies, 2008.

In some embodiments, the sample is a blood sample. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used.

In one embodiment, a sample may be obtained from an apparently healthy individual during a routine checkup and analyzed so as to provide an assessment of the individual's general health status. In another embodiment, a sample may be taken to screen for commonly occurring diseases. Such screening may encompass testing for a single disease, a family of related diseases or a general screening for multiple, unrelated diseases. Screening can be performed weekly, bi-weekly, monthly, bi-monthly, every several months, annually, or in several year intervals and may replace or complement existing screening modalities.

In another embodiment, an individual with a known increased probability of disease occurrence may be monitored regularly to detect for the appearance of a particular disease or class of diseases. An increased probability of disease occurrence can be based on familial association, age, previous genetic testing results, or occupational, environmental or therapeutic exposure to disease causing agents. Breast and ovarian cancer related to inherited mutations in the genes BRCA1 and BRCA2 are examples of diseases with a familial association wherein susceptible individuals can be identified through genetic testing. Another example is the presence of inherited mutations in the adenomatous polyposis coli gene predisposing individuals to colorectal cancer. Examples of environmental or therapeutic exposure include individuals occupationally exposed to benzene that have increased risk for the development of various forms of leukemia, and individuals therapeutically exposed to alkylating agents for the treatment of earlier malignancies. Individuals with increased risk for specific diseases can be monitored regularly for the first signs of an appearance of an abnormal discrete cell population. Monitoring can be performed weekly, bi-weekly, monthly, bi-monthly, every several months, annually, or in several year intervals, or any combination thereof. Monitoring may replace or complement existing screening modalities. Through routine monitoring, early detection of the presence of disease causative or associated cells may result in increased treatment options including treatments with lower toxicity and increased chance of disease control or cure.

In a further embodiment, testing can be performed to confirm or refute the presence of a suspected genetic or physiologic abnormality associated with increased risk of disease. Such testing methodologies can replace other confirmatory techniques like cytogenetic analysis or fluorescent in situ histochemistry (FISH). In still another embodiment, testing can be performed to confirm or refute a diagnosis of a pre-pathological or pathological condition.

In instances where an individual has a known pre-pathologic or pathologic condition, a plurality of discrete cell populations from the appropriate location can be sampled and analyzed to predict the response of the individual to available treatment options. In one embodiment, an individual treated with the intent to reduce in number or ablate cells that are causative or associated with a pre-pathological or pathological condition can be monitored to assess the decrease in such cells over time. A reduction in causative or associated cells may or may not be associated with the disappearance or lessening of disease symptoms. If the anticipated decrease in cell number does not occur, further treatment with the same or a different treatment regiment may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regiment can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of another discrete cell population(s) that are associated with a good prognosis. If a beneficial, discrete cell population is observed, measures can be taken to further increase their numbers, such as the administration of growth factors. Alternatively, individuals may be monitored for the appearance or increase in cell number of another discrete cell population(s) associated with a poor prognosis. In such a situation, renewed therapy can be considered including continuing, modifying the present therapy or initiating another type of therapy.

In these embodiments, one or more samples may be taken from the individual, and subjected to a modulator, as described herein. In some embodiments, the sample is divided into subsamples that are each subjected to a different modulator. After treatment with the modulator, different discrete cell populations in the sample or subsample are analyzed to determine their activation level(s). In some embodiments, single cells in the different discrete cell populations are analyzed. Any suitable form of analysis that allows a determination of cell activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. Determination of the activation level may be achieved by the use of activation state-specific binding elements, such as antibodies, as described herein. A plurality of activatable elements may be examined in one or more of the different discrete cell populations.

Certain fluid samples can be analyzed in their native state with or without the addition of a diluent or buffer. Alternatively, fluid samples may be further processed to obtain enriched or purified discrete cell populations prior to analysis. Numerous enrichment and purification methodologies for bodily fluids are known in the art. A common method to separate cells from plasma in whole blood is through centrifugation using heparinized tubes. By incorporating a density gradient, further separation of the lymphocytes from the red blood cells can be achieved. A variety of density gradient media are known in the art including sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells can be filtered out of diluted, whole blood following the lysis of red blood cells by using filters with pore sizes between 5 to 10 μm, as disclosed in U.S. patent application Ser. No. 09/790,673. Alternatively, whole blood can be separated into its constituent cells based on size, shape, deformability or surface receptors or surface antigens by the use of a microfluidic device as disclosed in U.S. patent application Ser. No. 10/529,453.

Select cell populations can also be enriched for or isolated from whole blood through positive or negative selection based on the binding of antibodies or other entities that recognize cell surface or cytoplasmic constituents. For example, U.S. Pat. No. 6,190,870 to Schmitz et al. discloses the enrichment of tumor cells from peripheral blood by magnetic sorting of tumor cells that are magnetically labeled with antibodies directed to tissue specific antigens.

Solid tissue samples may require the disruption of the extracellular matrix or tissue stroma and the release of single cells for analysis. Various techniques are known in the art including enzymatic and mechanical degradation employed separately or in combination. An example of enzymatic dissociation using collagenase and protease can be found in Wolters G H J et al. An analysis of the role of collagenase and protease in the enzymatic dissociation of the rat pancrease for islet isolation. Diabetologia 35:735-742, 1992. Examples of mechanical dissociation can be found in Singh, N P. Technical Note: A rapid method for the preparation of single-cell suspensions from solid tissues. Cytometry 31:229-232 (1998). Alternately, single cells may be removed from solid tissue through microdissection including laser capture microdissection as disclosed in Laser Capture Microdissection, Emmert-Buck, M. R. et al. Science, 274(8):998-1001, 1996.

In some embodiments, single cells can be analyzed within a tissue sample, such as a tissue section or slice, without requiring the release of individual cells before determining step is performed.

The cells can be separated from body samples by centrifugation, elutriation, density gradient separation, apheresis, affinity selection, panning, FACS, centrifugation with Hypaque, solid supports (magnetic beads, beads in columns, or other surfaces) with attached antibodies, etc. By using antibodies specific for markers identified with particular cell types, a relatively homogeneous population of cells may be obtained. Alternatively, a heterogeneous cell population can be used. Cells can also be separated by using filters. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Ser. Nos. 61/048,886; 61/048,920; and 61/048,657. See also, the commercial products from companies such as BD and BCI as identified above.

See also U.S. Pat. Nos. 7,381,535 and 7,393,656. All of the above patents and applications are incorporated by reference as stated above.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 30%.

### Determination of Activation State of a Discrete Cell Population

After treatment with one or more modulators, if used, in some embodiments the sample is analyzed to determine the activation state of different discrete cell populations. This generates activation state data of different discrete cell populations. In some embodiments, the activation state data of a discrete cell population is determined by contacting the cell population with one or more modulators and determining the activation state or activation level of an activatable element of at least one cell in the cell population. Different modulators suitable for use are outlined below in the section entitled “Modulators.” The activation level is determined by quantifying a relative amount of the activatable element in the cell (e.g. using antibodies to quantify the activatable element). As outlined in the section below entitled “Detection”, any suitable form of analysis that allows a determination of cell activation level(s) may be used. Activatable elements are described below in the section entitled “Activatable Elements.” Determination of the activation level may be achieved by the use of activation state-specific binding elements, such as antibodies, as described below in the sections entitled “Binding Elements” and “Alternative Activation State Indicators.” A plurality of activatable elements may be examined in one or more of the different discrete cell populations.

The population of cells can be divided into a plurality of samples, and the activation state data of the population is determined by measuring the activation level of at least one activatable element in the samples after the samples have been exposed to one or more modulators. In some embodiments, the analysis is performed in single cells. Any suitable analysis that allows determination of the activation level of an activatable element within single cells, which provides information useful for determining the activation state data of a discrete cell population from whom the sample was taken, may be used. Examples include flow cytometry, immunohistochemistry, immunofluorescent histochemistry with or without confocal microscopy, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, Inductively Coupled Plasma Mass Spectrometer (ICP-MS) and label-free cellular assays. Additional information for the further discrimination between single cells can be obtained by many methods known in the art including the determination of the presence of absence of extracellular and/or intracellular markers, the presence of metabolites, gene expression profiles, DNA sequence analysis, and karyotyping.

The activation state data of the different discrete cell populations can be used to understand communication between the discrete cell populations that are associated with disease. These causal associations may be determined using any suitable method known in the art, such as simple statistical test and/or classification algorithms. These causal associations may be modeled using Bayesian Networks or temporal models. Alternatively, these causal associations may be identified using unsupervised learning techniques such as principle components analysis and/or clustering. Causal association can be determined using activators or inhibitors that might affect one or more discrete cell populations. For example, an inhibitor that inhibits phosphorylation of an activatable element in a first cell population may have a causal effect on the phosphorylation of a second activatable element in a second cell population. In some embodiments, the causal association between discrete cell populations is already known in the art. Thus, in some embodiments, determining a causal association between discrete cell populations involves using associations already predetermined in the art. Causal associations between activation levels in different discrete cell populations may represent communications between cellular networks and can be used to determine the state of a cellular network. The state of a cellular network can be associated, for example, with drug response and disease progression.

a. Generation of Dynamic Activation State Data

In some embodiments, the activation levels of a discrete cell population or a discrete subpopulation of cells may be measured at multiple time intervals following treatment with a modulator to generate “dynamic activation state data” (also referred to herein as kinetic activation state data). In these embodiments, a sample or sub-sample (e.g. patient sample) is divided into aliquots which are then treated with one or more modulators. The different aliquots are then subject to treatment with a fixing agent at different time intervals. For instance, an aliquot that is to be measured at 5 minutes will be treated with one or more modulators and then subject to a treatment with a fixing agent after 5 minutes. The time intervals can vary greatly and will range from minutes (e.g. 5, 10, 15, 20, 25, 30, 35, 40, 45, 50, 55 minutes) to hours (e.g. 1, 2, 3, 4, 5, 6, 7, 8, 9, 10, 11, 12, 13, 14, 15, 6, 17 18, 19, 20, 21, 22, 23 hours) to days (e.g. 24 hours, 48 hours, 72 hours) or any combination thereof. Cells may also be treated with different concentrations of the modulator.

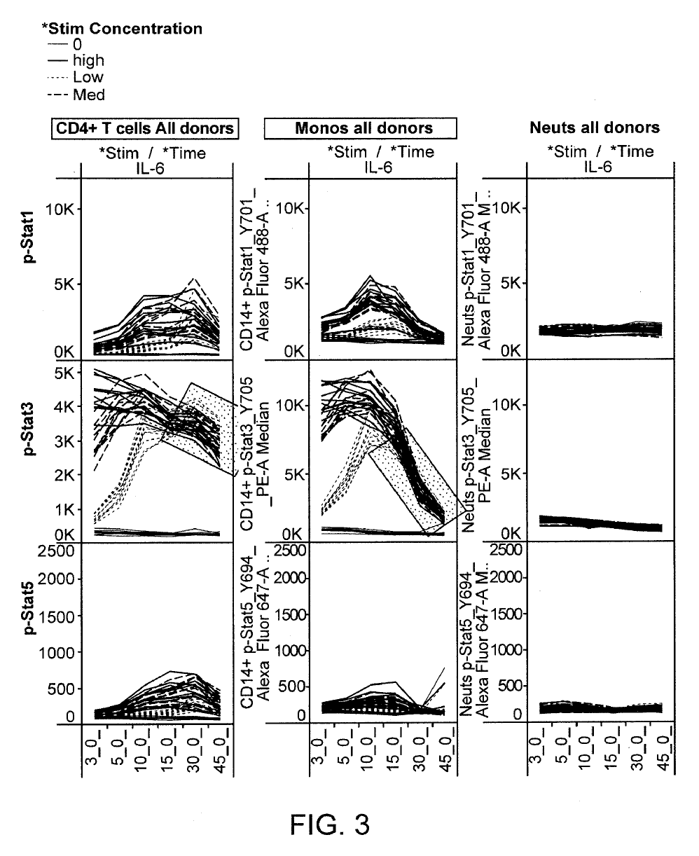

In these embodiments, the activation state data may be analyzed to identify discrete cell populations and then further analyzed to characterize the response of the different discrete cell populations to a modulator over time. The activation state data may be temporally modeled to characterize the dynamic response of the activatable elements to the stimulation with the modulator. Modeling the dynamic response to modulation can provide better understanding of the patho-physiology of a disease or prognostic status or a response to treatment. An example of modeling the dynamic response of normal cells to a modulator is shown in FIG. 3 and Example 6. Additionally, the modulator-induced activation levels of a discrete population of cells over time associated with a disease status may be compared of other samples to identify activation levels that represent an aberrant response to a modulator at specific time points. Aberrant response to a modulator may be associated with health status, a prognostic status, a cytogenetic status or predicted therapeutic response. Having activation levels at different time points is beneficial because the maximal differential response between samples associated with different statuses may be observed as early as 5 minutes after treatment with a modulator and as late as 72 hours after treatment with a modulator.

The modulator-induced response of the different discrete cell populations may be modeled to further understand communication between the discrete cell populations that are associated with disease. For example, an increased phosphorylation of an activatable element in a first cell population at an earlier time point may have a causal effect on the phosphorylation of a second activatable element in a second cell population at a later time point. These causal associations may be modeled using Bayesian Networks or temporal models. Alternatively, these causal associations may be identified using unsupervised learning techniques such as principle components analysis and/or clustering. Causal associations between activation levels in different discrete cell populations may represent communications between cellular networks over time. These communications may provide insight into the mechanism of drug response, cancer progression and carcinogenesis. Therefore, the identification and characterization of these communications allows for the development of diagnostics which can accurately predict drug response, therapeutic and early stage detection.

In some embodiments, the activation state data at a first time point is computationally analyzed (e.g. through binning or gating as described below) to determine discrete populations of cells. The discrete populations of cells are subsequently analyzed individually over the remaining time points to identify sub-populations of cells with different response to a modulator. Differential response over time within a same population of cells may be modeled using methods such as temporal modeling or hyper-spatial modeling as described in U.S. Patent Application 61/317,817 and below. These methods may allow the modeling of a single discrete cell population over time or multiple discrete cell populations over time.

In another embodiment, the activation state data is computationally analyzed at all of the time points to determine discrete populations of cells. The discrete populations of cells are then modeled in order to determine consistent membership in a discrete population of cells over time. In this way, the populations of cells are not characterized by the activation levels of modulators at a single time point, but rather are determined based on the activation levels of modulators at multiple time points. Both gating and binning may be used to first segregate the activation state data for cell populations at all of the time points. Based on the segregated cell populations at the various time points, discrete cell populations may be identified. Although this technique works well using gating or semi-supervised identification of discrete cell populations, this technique is ideal for use with unsupervised identification of discrete cell populations such as the methods described in U.S. Publication No. 2009/0307248 and below.

### Computational Identification of Discrete Populations of Cells

In some embodiments, the activation state data of a cell population is determined by contacting the cell population with one or more modulators, generating activation state data for the cell population and using computational techniques to identify one or more discrete cell populations based on the data. These techniques are implemented using computers comprising memory and hardware. In one embodiment, algorithms for generating metrics based on raw activation state data are stored in the memory of a computer and executed by a processor of a computer. These algorithms are used in conjunction with gating and binning algorithms, which are also stored and executed by a computer, to identify the discrete cell populations.

The data can be analyzed using various metrics. For example, the median fluorescence intensity (MFI) is computed for each activatable element from the intensity levels for the cells in the cell population gate. The MFI values are then used to compute a variety of metrics by comparing them to the various baseline or background values, e.g. the unstimulated condition, autofluorescence, and isotype control. The following metrics are examples of metrics that can be used in the methods described herein: 1) a metric that measures the difference in the log of the median fluorescence value between an unstimulated fluorochrome-antibody stained sample and a sample that has not been treated with a stimulant or stained (log(MFI_{Unstimulated Stained})−log(MFI_{Gated Unstained})), 2) a metric that measures the difference in the log of the median fluorescence value between a stimulated fluorochrome-antibody stained sample and a sample that has not been treated with a stimulant or stained (log(MFI_{stimulated Stained})−log(MFI_{Gated Unstained})), 3) a metric that measures the change between the stimulated fluorochrome-antibody stained sample and the unstimulated fluorochrome-antibody stained sample log(MFI_{stimulated Stained})−log(MFI_{Unstimulated Stained}), also called “fold change in median fluorescence intensity”, 4) a metric that measures the percentage of cells in a Quadrant Gate of a contour plot which measures multiple populations in one or more dimension 5) a metric that measures MFI of phosphor positive population to obtain percentage positivity above the background and 6) use of multimodality and spread metrics for large sample population and for subpopulation analysis.

In a specific embodiment, the equivalent number of reference fluorophores value (ERF) is generated. The ERF is a transformed value of the median fluorescent intensity values. The ERF value is computed using a calibration line determined by fitting observations of a standardized set of 8_peak rainbow beads for all fluorescent channels to standardized values assigned by the manufacturer. The ERF values for different samples can be combined in any way to generate different activation state metric. Different metrics can include: 1) a fold value based on ERF values for samples that have been treated with a modulator (ERFₘ) and samples that have not been treated with a modulator (ERFᵤ), log₂ (ERFₘ/ERFᵤ); 2) a total phospho value based on ERF values for samples that have been treated with a modulator (ERFₘ) and samples from autofluorecsent wells (ERFₐ), log₂ (ERFₘ/ERFₐ); 3) a basal value based on ERF values for samples that have not been treated with a modulator (ERFᵤ) and samples from autofluorescent wells (ERFₐ), log₂ (ERFᵤ/ERFₐ); 4) A Mann-Whitney statistic Uᵤ comparing the ERF_{m and} ERFᵤ values that has been scaled down to a unit interval (0,1) allowing inter-sample comparisons; 5) A Mann-Whitney statistic Uᵤ comparing the ERF_{m and} ERFᵤ values that has been scaled down to a unit interval (0,1) allowing inter-sample comparisons; 5) a Mann-Whitney statistic Uₐ comparing the ERFₐ and ERFₘ values that has been scaled down to a unit interval (0,1); and 6) A Mann-Whitney statistic U75. U75 is a linear rank statistic designed to identify a shift in the upper quartile of the distribution of ERFₘ and ERFᵤ values. ERF values at or below the 75^{th} percentile of the ERFₘ and ERFᵤ values are assigned a score of 0. The remaining ERFₘ and ERFᵤ values are assigned values between 0 and 1 as in the Uᵤ statistic. For activatable elements that are surface markers on cells, the following metrics may be further generated: 1) a relative protein expression metric log 2(ERFₛₜₐᵢₙ)−log 2(ERF_{control}) based on the ERF value for a stained sample (ERFₛₜₐᵢₙ) and the ERF value for a control sample (ERF_{control}); and 2) A Mann-Whitney statistic Ui comparing the ERFₘ and ERFᵢ values that has been scaled down to a unit interval (0,1), where the ERF, values are derived from an isotype control.

The activation state data for the different markers is “gated” in order to identify discrete subpopulations of cells within the data. In gating, activation state data is used to identify discrete sub-populations of cells with distinct activation levels of an activatable element. These discrete sub-populations of cells can correspond to cell types, cell sub-types, cells in a disease or other physiological state and/or a population of cells having any characteristic in common

In some embodiments, the activation state data is displayed as a two-dimensional scatter-plot and the discrete subpopulations are “gated” or demarcated within the scatter-plot. According to the embodiment, the discrete subpopulations may be gated automatically, manually or using some combination of automatic and manual gating methods. In some embodiments, a user can create or manually adjust the demarcations or “gates” to generate new discrete sub-populations of cells. Suitable methods of gating discrete sub-populations of cells are described in U.S. patent application Ser. No. 12/501,295, the entirety of which is incorporated by reference herein, for all purposes.

In some embodiments, the discrete cell populations are gated according to markers that are known to segregate different cell types or cell sub-types. In a specific embodiment, a user can identify discrete cell populations based on surface markers. For example, the user could look at: “stem cell populations” by CD34+ CD38− or CD34+ CD33− expressing cells; memory CD4 T lymphocytes; e.g. CD4⁺CD45RA⁺CD29^{low} cells; or multiple leukemic sub-clones based on CD33, CD45, HLA-DR, CD11b and analyzing signaling in each discrete population/subpopulation. In another alternative embodiment, a user may identify discrete cell populations/subpopulations based on intracellular markers, such as transcription factors or other intracellular proteins; based on a functional assay (e.g., dye efflux assay to determine drug transporter+cells or fluorescent glucose uptake) or based on other fluorescent markers. In some embodiments, gates are used to identify the presence of specific discrete populations and/or subpopulations in existing independent data. The existing independent data can be data stored in a computer from a previous patient, or data from independent studies using different patients.

In some embodiments, the discrete cell populations/subpopulations are automatically gated according to activation state data that segregates the cells into discrete populations. For example, an activatable element that is “on” or “off” in cells may be used to segregate the cell population into two discrete subpopulations. In embodiments where the discrete cell subpopulations are automatically identified, different algorithm may be used to identify discrete cell subpopulations based on the activation state data. In a specific embodiment, a multi-resolution binning algorithm is used to iteratively identify discrete subpopulations of cell by partitioning the activation state data. This algorithm is outlined in detail in U.S. Publication No. 2009/0307248, which is incorporated herein in its entirety, for all purposes. In one embodiment, the multi-resolution binning algorithm is used to identify rare or uniquely discrete cell populations by iteratively identifying vectors or “hyperplanes” that partition activation state data into finer resolution bins. Using iterative algorithms such as multi-resolution binning algorithms, fine resolution bins containing rare populations of cells may be identified. For example, activation state data for one or more markers may be iteratively binned to identify a small number of cells with an unusually high expression of a marker. Normally, these cells would be discarded as “outlier” data or during normalization of the data. However, multi-resolution binning allows the identification of activation state data corresponding to rare populations of cells.

In different embodiments, gating can be used in different ways to identify discrete cell populations. In one embodiment, “Outside-in” comparison of activation state data for individual samples or subset (e.g., patients in a trial) is used to identify discrete cell populations. In this embodiment, cell populations are homogenous or lineage gated in such a way as to create discrete sets of cells considered to be homogenous based on a characteristic (e.g. cell type, expression, subtype, etc.). An example of sample-level comparison in an AML patient would be the identification of signaling profiles in lymphocytes (e.g., CD4 T cells, CD8 T cells and/or B cells), monocytes+granulocytes and leukemic blast and correlating the activation state data of these populations with non-random distribution of clinical responses. This is considered an outside-in approach because the discrete cell population of interest is pre-defined prior to the mapping and comparison of its profile to, e.g., a clinical outcome or the profile of the populations in normal individuals.

In other embodiments, “Inside-out” comparison of activation state data at the level of individual cells in a heterogeneous population is used to identify discrete cell populations. An example of this would be the signal transduction state mapping of mixed hematopoietic cells under certain conditions and subsequent comparison of computationally identified cell clusters with lineage specific markers. This could be considered an inside-out approach to single cell studies as it does not presume the existence of specific discrete cell populations prior to classification. Suitable methods for inside-out identification of discrete cell populations include the multi-resolution binning algorithm described above. A major drawback of this approach is that it creates discrete cell populations which, at least initially, require multiple transient markers to enumerate and may never be accessible with a single cell surface epitope. As a result, the biological significance of such discrete cell populations can be difficult to determine. The main advantage of this unconventional approach is the unbiased tracking of discrete cell populations without drawing potentially arbitrary distinctions between lineages or cell types and the potential of using the activation state data of the different populations to determine the status of an individual.

Each of these techniques capitalizes on the ability of flow cytometry to deliver large amounts of multi-parametric data at the single cell level. For discrete cell populations associated with a condition (e.g. neoplastic or hematopoetic condition), a third “meta-level” of data exists because cells associated with a condition (e.g. cancer cells) are generally treated as a single entity and classified according to historical techniques. These techniques have included organ or tissue of origin, degree of differentiation, proliferation index, metastatic spread, and genetic or metabolic data regarding the patient.

In some embodiments, the present invention uses variance mapping techniques for mapping condition signaling space. These methods represent a significant advance in the study of condition biology because it enables comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and reclusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g. two tumors) that are thought to be relatively alike based on lineage markers or tissue of origin could have vastly different abilities to interpret environmental stimuli and would be profiled in two different categories.

Classifying and Characterizing Cell Network Based on Activation State Data Associated with Discrete Populations of Cells

When the activation state data associated with a plurality of discrete cell populations has been identified, it is frequently useful to determine whether activation state data is non-randomly distributed within the categories such as disease status, therapeutic response, clinical responses, presence of gene mutations, and protein expression levels. Activation state data that are strongly associated with one or more discrete cell populations with a specific characteristic (e.g. gene mutation, disease status) can be used both to classify a cell according to the characteristic and to further characterize and understand the cell network communications underlying the pathophysiology of the characteristic. Activation state data that uniquely identifies a discrete cell populations associated with a cell network can serve to re-enforce or complement other activation state data that uniquely identifies another discrete cell population associated with the cell network.

If activation state data is available for many discrete cell populations, activation state data that uniquely identifies a discrete cell population may be identified using simple statistical tests, such as the Student's t-test and the X² test. Similarly, if the activation state data of two discrete cell populations within the experiment is thought to be related, the r² correlation coefficient from a linear regression can used to represent the degree of this relationship. Other methods include Pearson and Spearman rank correlation. In some embodiment, correlation and statistical test algorithms will be stored in the memory of a computer and executed by a processor associated with the computer.

In some embodiments, the invention provides methods for determining whether the activation state data of different discrete cell populations is associated with a cellular network and/or a characteristic that can potentially complement each other to improve the accuracy of classification. In these embodiments, the activation state data of the discrete cell populations may be used generate a classifier for one or more characteristics associated with the discrete cell populations including but not limited to: therapeutic response, disease status and disease prognosis. A classifier, as defined herein, is any type of statistical model that can be used to characterize a similarity between a sample and a class of samples. Classifiers can comprise binary and multi-class classifiers as in the traditional use of the term classifier. Classifiers can also comprise statistical models of activation levels and variance in only one class of samples (e.g. normal individuals). These single-class classifiers may be applied to data, e.g., from undiagnosed samples, to produce a similarity value, which can be used to determine whether the undiagnosed sample belongs to the class of samples (e.g. by using a threshold similarity value). Any suitable method known in the art can be used to generate the classifier. For example, simple statistical tests can be used to generate a classifier. Examples, of classification algorithms that can be used to generate a classifier include, but are not limited to, Linear classifiers, Fisher's linear discriminant, ANOVA, Logistic regression, Naive Bayes classifier, Perceptron, Support vector machines, Quadratic classifiers, Kernel estimation, k-nearest neighbor, Boosting. Decision trees, Random forests, Neural networks, Bayesian networks, Hidden Markov models, and Learning vector quantization. Thus, in some embodiments, different types of classification algorithms may be used to generate the classifier including but not limited to: neural networks, support vector machines (SVMs), bagging, boosting and logistic regression. In some embodiments, the activation state data for different discrete populations associated with a same network and/or characteristic may be pooled before generating a classifier that specifies which combinations of activation state data associated with discrete cell populations can be used to uniquely identify and classify cells according to the activatable element.

In a specific embodiment, if the size of the activation state data associated with the discrete populations of cells is small, a straightforward corner classifier approach for picking combinations of activation state data that uniquely identifies the different discrete cell populations can be adopted. Combinations of discrete cell populations' activation state data can also be tested for their stability via a bootstrapping approach described below. In this embodiment, a corners classification algorithm with be applied to the data. The corners classifier is a rules-based algorithm for dividing subjects into two classes (e.g. dichotomized response to a treatment) using one or more numeric variables (e.g. population/node combination). This method works by setting a threshold on each variable, and then combining the resulting intervals (e.g., X<10, or Y>50) with the conjunction (and) operator (reference). This creates a rectangular region that is expected to hold most members of the class previously identified as the target (e.g. responders or non-responders of treatment). Threshold values are chosen by minimizing an error criterion based on the logit-transformed misclassification rate within each class. The method assumes only that the two classes (e.g. response or lack of response to treatment) tend to have different locations along the variables used, and is invariant under monotone transformations of those variables.

In some embodiments, computational methods of cross-validation are used during classifier generation to measure the accuracy of the classifier and prevent over-fitting of the classifier to the data. In a specific embodiment, bagging techniques, aka bootstrapped aggregation, are used to internally cross-validate the results of the above statistical model. In this embodiment, re-samples are iteratively drawn from the original data and used to validate the classifier. Each classifier, e.g. combination of population/node, is fit to the resample, and used to predict the class membership of those patients who were excluded from the resample. The accuracy of false positive and false negative classifications is determined for each classifier.

After iteratively re-sampling the original data, each patient acquires a list of predicted class memberships based on classifiers that were fit using other patients. Each patient's list is reduced to the fraction of target class predictions; members of the target class should have fractions near 1, unlike members of the other class. The set of such fractions, along with the patient's true class membership, is used to create a Receiver Operator Curve and to calculate the area under the ROC curve (herein referred to as the “AUC”).

In some embodiments, the invention provides methods for determining a status of an individual such a disease status, therapeutic response, and/or clinical responses wherein the positive predictive value (PPV) is higher than 60, 70, 80, 90, 95, or 99.9%. In some embodiments, the invention provides methods for determining a status of an individual such a disease status, therapeutic response, and/or clinical responses, wherein the PPV is equal or higher than 95%. In some embodiments, the invention provides methods determining a status of an individual such a disease status, therapeutic response, and/or clinical responses, wherein the negative predictive value (NPV) is higher than 60, 70, 80, 90, 95, or 99.9%. In some embodiments, the invention provides methods for determining a status of an individual such a disease status, therapeutic response, and/or clinical responses, wherein the NPV is higher than 85%.

In some embodiments, the invention provides methods for predicting risk of relapse at 2 years, wherein the PPV is higher than 60, 70, 80, 90, 95, or 99.9%. In some embodiments, the invention provides methods for predicting risk of relapse at 2 years, wherein the PPV is equal or higher than 95%. In some embodiments, the invention provides methods for predicting risk of relapse at 2 years, wherein the NPV is higher than 60, 70, 80, 90, 95, or 99.9%. In some embodiments, the invention provides methods for predicting risk of relapse at 2 years, wherein the NPV is higher than 80%. In some embodiments, the invention provides methods for predicting risk of relapse at 5 years, wherein the PPV is higher than 60, 70, 80, 90, 95, or 99.9%. In some embodiments, the invention provides methods for predicting risk of relapse at 5 years, wherein the PPV is equal or higher than 95%. In some embodiments, the invention provides methods for predicting risk of relapse at 5 years, wherein the NPV is higher than 60, 70, 80, 90, 95, or 99.9%. In some embodiments, the invention provides methods for predicting risk of relapse at 5 years, wherein the NPV is higher than 80%. In some embodiments, the invention provides methods for predicting risk of relapse at 10 years, wherein the PPV is higher than 60, 70, 80, 90, 95, or 99.9%. In some embodiments, the invention provides methods for predicting risk of relapse at 10 years, wherein the PPV is equal or higher than 95%. In some embodiments, the invention provides methods for predicting risk of relapse at 10 years, wherein the NPV is higher than 60, 70, 80, 90, 95, or 99.9%. In some embodiments, the invention provides methods for predicting risk of relapse at 10 years, wherein the NPV is higher than 80%.

In some embodiments, the p value in the analysis of the methods described herein is below 0.05, 04, 0.03, 0.02, 0.01, 0.009, 0.005, or 0.001. In some embodiments, the p value is below 0.001. Thus in some embodiments, the invention provides methods for determining a status of an individual such a disease status, therapeutic response, and/or clinical responses, wherein the p value is below 0.05, 04, 0.03, 0.02, 0.01, 0.009, 0.005, or 0.001. In some embodiments, the p value is below 0.001. In some embodiments, the invention provides methods for determining a status of an individual such a disease status, therapeutic response, and/or clinical responses, wherein the AUC value is higher than 0.5, 0.6, 07, 0.8 or 0.9. In some embodiments, the invention provides methods for determining a status of an individual such a disease status, therapeutic response, and/or clinical responses, wherein the AUC value is higher than 0.7. In some embodiments, the invention provides methods for determining a status of an individual such a disease status, therapeutic response, and/or clinical responses, wherein the AUC value is higher than 0.8. In some embodiments, the invention provides methods for determining a status of an individual such a disease status, therapeutic response, and/or clinical responses, wherein the AUC value is higher than 0.9.

In another embodiment, activation state data generated for a cellular network over a series of time points may be used to identify activation state data that represents unique communications within the cellular network over time. The activation state data that represents unique communications within the cellular network can be used to classify other activation state data associated with cell populations to determine whether they are associated with a same characteristic as the cellular network or determine if there are in a specific stage or phase in time that is unique to a cellular network. For example, different discrete populations of cells in a cellular network may be treated with a same modulator and sub-sampled over a series of time points to determine communications between the discrete populations of cells that are unique to the stimulation with the modulator. Similarly, samples of different discrete cell populations may be derived from patients over the course of treatment and used to identify communications between the discrete populations of cells that are unique to the course of treatment.

In one embodiment, the activation state data for the discrete cell populations at different time points may be modeled to represent dynamic interactions between the discrete cell populations in a cell networks over time. The activation state data may be modeled using temporal models, Bayesian networks or some combination therefore. Suitable methods of generating Bayesian networks are described in 11/338,957, the entirety of which is incorporated herein, for all purposes. Suitable methods of generating temporal models of activation state data are described in U.S. Patent Application 61/317,817, the entirety of which is incorporated herein by reference. Different metrics may be generated to describe the dynamic interactions including: derivatives, integrals, rate-of-change metrics, splines, state representations of activation state data and Boolean representations of activation state data.

In embodiments where metrics and other values describing dynamic interactions are generated, these values and metrics are used to generate a classifier. As outlined above, any suitable classification algorithm can be used to determine metrics and values that uniquely identify cellular network data that shares a same characteristic. In some embodiments, the descriptive values and metrics will be generated based on two distinct data sets: 1) activation state data that is associated with a characteristic and 2) activation state data that is not association with a characteristic. For example: activation state data generated from discrete cell populations after stimulation with a modulator and activation state data generated from un-stimulated discrete cell populations. In these embodiments, the descriptive values and metrics will be used to generate a two-class classifier. In other embodiments, descriptive values and metrics will be generated from a large number of activation state data sets associated with different characteristics and a multi-class classifier will be generated. The resulting classifier will be used to determine whether a cellular network is part of the data set.

In some embodiments, the above classifiers are used to characterize activation state data derived from an individual such as a patient. In these embodiments, activation state data associated with a cellular network of one or more discrete cell populations is derived from a patient. In some embodiments, the activation state data associated with the different discrete cell populations from a patient may be identified by obtaining patient samples with different characteristics (e.g. blood cells and tumor samples). In some embodiments, the activation state data associated with the different discrete cell populations may be identified computationally based on activation state data for activatable elements that are known to differentiate discrete cell populations. A classifier that specifies activation state data from different discrete cell populations used to determine whether the cells have a common characteristic is applied to the activation state data associated with the individual in order to generate a classification value that specifies the probability that the individual (or the cells derived from the individual) is associated with the characteristic. In most embodiments, the classifier is stored in computer memory or computer-readable storage media as a set of values or executable code and applying the classifier comprises executing code that applies the classifier to the activation state data associated with the individual. The classification value may be output to a user, transmit to an entity requesting the classification value and/or stored in memory associated with a computer. The classification value may represent information related to or representing the physiological status of the individual such as a diagnosis, a prognosis or a predicted response to treatment.

In some embodiments, the activation state data of a plurality of cell populations is determined in normal individuals or individual not suffering or not suspected of suffering from a condition. This activation state data can be used to create statistical model of the ranges of activation levels observed in cell populations derived from samples obtained from normal patients (e.g. regression model, variance model). This ranges and/or models may be used to determine whether samples from undiagnosed individuals exhibit the range of activation state data observed in normal samples (e.g., range of normal activation levels). This can be used to create a classifier for normal individuals. In some embodiments, the models may be used to generate a similarity value that indicates the similarity of the activation state data associated with the undiagnosed individual to the range of normal activation levels (e.g. correlation coefficient, fitting metric) and/or a probability value that indicates the probability that the activation state data would be similar to the range of normal activation levels by chance (i.e. probability value and/or associated confidence value). In other embodiments, activation state data from normal patients may be combined with activation state data from patients that are known to have a disease to create a binary or multi-class classifier. In some embodiments, the activation state data from an undiagnosed individual will be displayed graphically with the range of activation states observed in normal cells. This allows for a person, for example a physician, to visually assess the similarity of the activation state data associated with the undiagnosed patient to that range of activation states observed in samples from normal individuals. Examples of how to create statistical models or profiles of the ranges of activation levels observed in cell populations derived from samples obtained from normal patients and their uses in classifying individual are described in US provisional entitled “Benchmarks for Normal Cell Identification” filed Sep. 8, 2010 with attorney docket number 134.001, the entirety of which is incorporated by reference herein for all purposes.

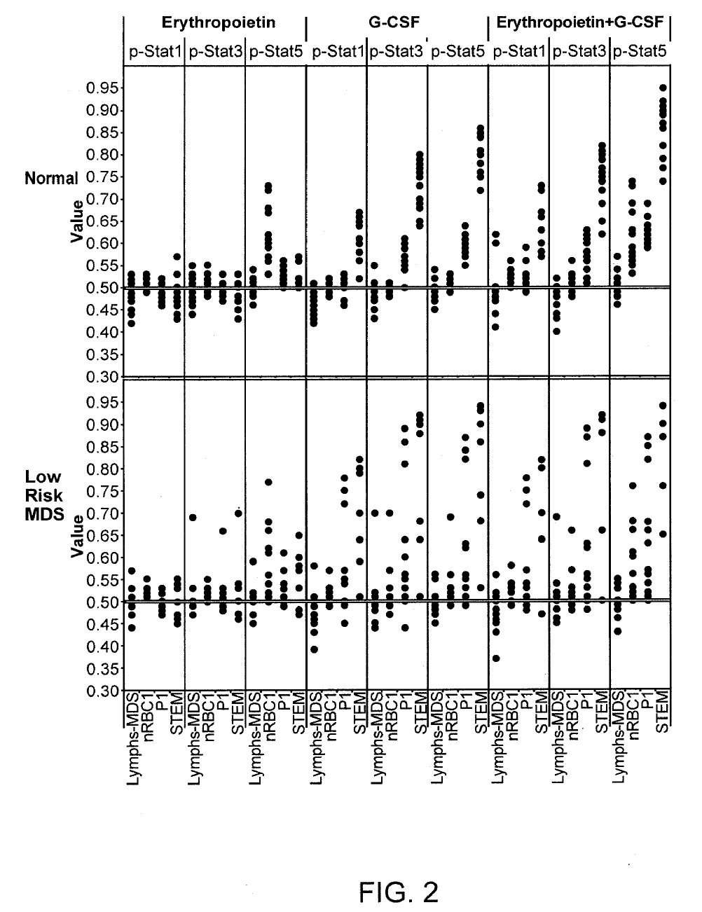

In some embodiments, the present invention includes method for evaluating cells that may be cancerous. The cells are subjected to the methods described herein and compared to a population of normal cells. The comparison can be done with any of the algorithms described herein. In some embodiments, the activation state data is represented in graphical form. Typically, when shown in a graph, normal cells have a uniform population and appear tightly grouped with narrow boundaries. When cancerous or pre-cancerous cells are subject to the same methods as normal cells (e.g., treatment with one or more modulators) and are represented on the same graph, deviations from the norm shown by the graph indicate a more heterogeneous population. An example is illustrated in FIG. 2 and Example 5. This change is an indication that the cells may be cancerous in a manner that is a function of the degree of change. Morphology change may indicate a cancerous population on a continuation from mild to metastatic. If there is no shape change from normal, then there may not be a change in the cell phenotype.

The presence of a heterogeneous population of cells may indicate that therapy is needed. The outcome of the therapy can be monitored by reference to the graph. A change from a more heterogeneous population to a population that is more tightly grouped on the chart may indicate that the cell population is returning to a normal state. The lack of change may indicate that the therapy is not working and the cell population is refractory or resistant to therapy. It may also indicate that a different discrete cell population has changed over to the cancerous phenotype. Lack of change back to normal is indicative of a negative correlation to therapy. These changes may be genetic or epigenetic.

One embodiment of the present invention is to conduct the methods described herein by analyzing a population of normal cells to create a pattern or a database that can be compared in a graphical way to a cell population that is potentially cancerous. The analysis can be by many methods, but one preferred method is the use of flow cytometry.

In all these embodiments, the activation state data may be generated at a central laboratory and the classifier may be applied to the data at the central laboratory. Alternately, the activation state data may be generate by a third party and transmitted, for example, via a secure network to a central laboratory for classification. Methods of transmitting data for classification and analysis are outlined in U.S. patent application Ser. No. 12/688,851, the entirety of which is incorporated herein by reference, for all purposes.

### Methods

In some embodiments, this invention is directed to methods and compositions, and kits that allow for the determination of the status of an individual and/or the state of a cellular network comprised of at least two discrete cell populations. The methods and compositions, and kits described herein for any condition for which a correlation between the condition, its prognosis, course of treatment, or other relevant characteristic, and the state of a cellular network and/or activation state data of a plurality of cell populations, e.g., activation level of one or more activatable elements in the populations, in samples from individuals may be ascertained. In some embodiments, this invention is directed to methods and compositions, and kits for analysis, drug screening, diagnosis, prognosis, for methods of disease treatment and prediction. In some embodiments, the present invention involves methods of analyzing experimental data. In some embodiments, the activation state data of different discrete cell populations in a sample (e.g. clinical sample) is used, e.g., in diagnosis or prognosis of a condition, patient selection for therapy using some of the agents identified above, to monitor treatment, modify therapeutic regimens, and/or to further optimize the selection of therapeutic agents which may be administered as one or a combination of agents. Hence, therapeutic regimens can be individualized and tailored according to the data obtained prior to, and at different times over the course of treatment, thereby providing a regimen that is individually appropriate. In some embodiments, a compound is contacted with cells to analyze the response to the compound. The activation state data of a discrete cell population can be generated by quantifying the activation level of at least one activatable element in response to at least one modulator in one or more cells belonging to the cell population.

The invention allows for the determination of the state of a cellular network comprising two or more discrete cell populations. The methods of the invention provide tools useful in the treatment of an individual afflicted with a condition, including but not limited to: methods for assigning a risk group, methods of predicting an increased risk of relapse, methods of predicting an increased risk of developing secondary complications, methods of choosing a therapy for an individual, methods of predicting duration of response, response to a therapy for an individual, methods of determining the efficacy of a therapy in an individual, and methods of determining the prognosis for an individual. The state of a cellular network can serve as a prognostic indicator to predict the course of a condition, e.g. whether the course of a neoplastic or a hematopoietic condition in an individual will be aggressive or indolent, thereby aiding the clinician in managing the patient and evaluating the modality of treatment to be used. In another embodiment, the present invention provides information to a physician to aid in the clinical management of a patient so that the information may be translated into action, including treatment, prognosis or prediction.

In some embodiments, the methods described herein are used to screen candidate compounds useful in the treatment of a condition or to identify new drug targets.

In some embodiments, the status of the individual or the state of the cellular network can be used to confirm or refute the presence of a suspected genetic or physiologic abnormality associated with increased risk of disease. Such testing methodologies can replace other confirmatory techniques like cytogenetic analysis or fluorescent in situ histochemistry (FISH). In still another embodiment, the status of the individual or the state of the cellular network can be used to confirm or refute a diagnosis of a pre-pathological or pathological condition.

In instances where an individual has a known pre-pathologic or pathologic condition, the status of the individual or the state of the cellular network can be used to predict the response of the individual to available treatment options. In one embodiment, an individual treated with the intent to reduce in number or ablate cells that are causative or associated with a pre-pathological or pathological condition can be monitored to assess the decrease in such cells and the state of a cellular network over time. A reduction in causative or associated cells may or may not be associated with the disappearance or lessening of disease symptoms, e.g. depending of the state of the cellular network. If the anticipated decrease in cell number and/or improvement in the state of a cellular network do not occur, further treatment with the same or a different treatment regiment may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regiment can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of a discrete cell population(s) that are associated with a good prognosis. If a beneficial discrete population of cells is observed, measures can be taken to further increase their numbers, such as the administration of growth factors. Alternatively, individuals may be monitored for the appearance or increase in cell number of a discrete cells population(s) associated with a poor prognosis. In such a situation, renewed therapy can be considered including continuing, modifying the present therapy or initiating another type of therapy.

In some embodiments, the determination of the status of an individual may be used to ascertain whether a previous condition or treatment has induced a new pre-pathological or pathological condition that requires monitoring and/or treatment. For example, treatment for many forms of cancers (e.g. lymphomas and childhood leukemias) can induce certain adult leukemias, and the methods of the present invention allow for the early detection and treatment of such leukemias.

The invention provides methods for determining characteristics such as the disease status of an individual by analyzing different discrete cell populations in said individual. In some embodiments, the disease status of an individual is determined by a method comprising contacting a first cell from a first discrete cell population from said individual with at least a first modulator, contacting a second cell from a second discrete cell population from said individual with at least a second modulator, determining an activation level of at least one activatable element in said first cell and said second cell, creating a response panel for said individual comprising said determined activation levels of said activatable elements, and making a decision regarding the disease status of said individual, wherein said decision is based on said response panel.

In some embodiments, one or more samples containing the different discrete cell populations may be taken from the individual, and subjected to a modulator, as described herein. In some embodiments, the sample is divided into subsamples that are each subjected to a different modulator. After treatment with the modulator, different discrete populations of cells in the sample or subsample are analyzed to determine their activation level(s). In some embodiments, single cells in the different discrete cell populations are analyzed. Any suitable form of analysis that allows a determination of activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. Determination of the activation level may be achieved by the use of activation state-specific binding elements, such as antibodies, as described herein. A plurality of activatable elements may be examined in one or more of the different discrete cell populations.

In some embodiments, the invention provides methods for determining the status of a cellular network in an individual by analyzing different discrete cell populations in said individual. The analysis of different discrete cell populations allows for the determination of directionality (e.g. vectors) within the different discrete cell populations participating in a cellular network. The analysis of the different discrete cell populations can be performed by determining the activation level of at least one activatable element in the different discrete cell populations in response to a modulator. In some embodiments, the analysis of the different discrete cell populations is performed by dividing each discrete cell population into a plurality of samples and determining the activation level of at least one activatable element in the samples in response to a modulator.

In some embodiments, the invention is directed to methods of determining the presence or absence of a condition in an individual by subjecting a plurality of different discrete cell populations from the individual to a modulator, determining the activation level of an activatable element in the a plurality of different discrete cell populations, and determining the presence or absence of the condition based on the activation level upon treatment with a modulator. In some embodiments, each discrete cell population is contacted with a different modulator in separate cultures. In some embodiments, each discrete cell population is contacted with the same modulator in the same or separate cultures. The term “same modulator” as described herein in relation to a modulator encompasses active fragment or portion of the modulator, a modulator that binds the same target as the modulator and/or a modulator that modulates the same signaling pathway as the modulator. For example, when a discrete cell population is treated with a modulator as described herein, another discrete cell population treated with the same modulator can be treated with an active fragment or portion of the modulator, a modulator that binds the same target and/or a modulator that modulates the same signaling pathway. In some embodiments, some discrete cell populations are contacted with the same modulator in the same or separate cultures, while other discrete cell populations are contacted with a different modulator. In some embodiments, the contacting of discrete cell population is before isolation of said first cell and said second cell from said individual, for example, when the modulator such as a chemical is in the cell environment inside of the individual. Thus, in some embodiments the modulator is present inside the individual and the discrete cell populations are contacted by the modulator in a cell environment inside the individual.

In some embodiments, the determination of status of a cellular network comprises the detection and determination of the activation state of immune cells specifically related to the pathogenesis of autoimmune diseases. Specific immune cells can be monitored over time while they are under therapeutic pressure either in vitro or in vivo to provide information to guide patient management.

In some embodiments, the invention provides methods for determining a status of an individual such a disease status, therapeutic response, and/or clinical responses wherein the positive predictive value (PPV) is higher than 60, 70, 80, 90, 95, or 99.9%. In some embodiments, the invention provides methods for determining a status of an individual such a disease status, therapeutic response, and/or clinical responses, wherein the PPV is equal or higher than 95%. In some embodiments, the invention provides methods determining a status of an individual such a disease status, therapeutic response, and/or clinical responses, wherein the negative predictive value (NPV) is higher than 60, 70, 80, 90, 95, or 99.9%. In some embodiments, the invention provides methods for determining a status of an individual such a disease status, therapeutic response, and/or clinical responses, wherein the NPV is higher than 85%.

In some embodiments, the p value in the analysis of the methods described herein is below 0.05, 04, 0.03, 0.02, 0.01, 0.009, 0.005, or 0.001. In some embodiments, the invention provides methods for determining a status of an individual such a disease status, therapeutic response, and/or clinical responses, wherein the AUC value is higher than 0.5, 0.6, 07, 0.8 or 0.9.

In some embodiments, a discrete population of cells is a population of cells wherein every cell has the same or substantially the same of a set of extracellular markers or range of extracellular markers that are used to identify the discrete cell population. The set of extracellular markers can be one extracellular marker. For example, “stem cell populations” are characterized by CD34+ CD38− or CD34+ CD33− expressing cells, memory CD4 T lymphocytes by CD4⁺CD45RA⁺CD29^{low} cells, and multiple leukemic subclones can be identified based on CD33, CD45, HLA-DR, CD11b. In addition to extracellular markers, expression levels of intracellular biomolecules, e.g., proteins, may be used alone or in combination with the extracellular markers to identify a cell population. Further, additional cellular elements, e.g., biomolecules or molecular complexes such as RNA, DNA, carbohydrates, metabolites, and the like, may be used in conjunction with extracellular markers and/or expression levels in the identification of cell populations encompassed here.

In some embodiments, other biological processes that affect the status of a cellular constituent may also be used to identify a cell population. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

A discrete population of cells, additionally, may be further divided into subpopulations that are themselves discrete cell populations based on other factors, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, if B cells represent a predefined class, they can be further subdivided based on the expression of cell surface markers such as CD19, CD20, or CD22.

Alternatively, a discrete population of cells can be aggregated based upon shared characteristics that may include inclusion in one or more additional discrete cell populations or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing characteristics.

The absence of a discrete subpopulation of cells is itself activation state data that is useful in understanding the pathophysiology of a discrete population of cells. This is useful, for example, when it is desired to determine what the percentage of the total number of a discrete population of cells belongs to one particular subpopulation of cells.

The discrete populations of cells may be identified based on empirical characteristics derived from individuals that indicate the status of individuals, e.g., health status. For example, blood samples from the clinic and/or from clinical trials may be analyzed retrospectively to identify discrete populations of cells; the activation state data of certain populations or quantitative features of the discrete cell populations may be associated with certain known outcomes for the patients.

For example, blood samples may be obtained from cancer patients over the course of treatment. Various outcomes, from complete remission for a number of years, to death from cancer or cancer recurrence after treatment, may be recorded. Profiles of the states of activatable elements in a plurality of discrete cell populations, with or without modulator, may be obtained from retrospective samples to determine discrete populations of cells present in the samples, activation state data in each discrete population of cells, numbers of cells in each discrete population of cells, relative numbers or proportions of cells in different discrete populations and/or subpopulations of cells, and the like. These discrete populations of cells together with their predictive value for various health status, may be placed in a database that is then used for analysis of further samples. As more samples are obtained and correlated health status determined, the database may be modified.

In some embodiments the different discrete cell populations are hematopoietic cell populations. Examples of hematopoietic populations include, but are not limited to, pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells. Thus, for example, in some embodiments, the status of an individual is determined by analyzing the activation level of an activatable element in a B-lymphocyte-derived discrete cell population and a T-lymphocyte-derived discrete cell population in response to a modulator, wherein the modulator for the different discrete cell populations can be the same or different.

In some embodiments, the different discrete cell populations are subpopulations of a discrete population of cells. For example, in some embodiments where the discrete populations of cells are hematopoietic cell populations, the status of an individual is determined by analyzing the activation level of an activatable element in a naive B-lymphocyte discrete cell population and a memory B-lymphocyte discrete cell population in response to a modulator, wherein the modulator for the different discrete cell population can be the same or different. In another example, in some embodiments, in some embodiments, the status of an individual is determined by analyzing the activation level of an activatable element in a CD4⁺ T-lymphocyte population and a CD8⁺ T-lymphocyte derived population in response to a modulator, wherein the modulator for the different discrete cell population can be the same or different.

In some embodiments, the status of an individual or the state of cellular network is determined by creating a response panel by analyzing one or more activatable elements in different discrete cell populations in response to one or more modulators. In some embodiments, a response panel is created by contacting each of the different discrete cell populations with at least one modulator and determining an activation level of at least one activatable element in each of the discrete cell populations. In some embodiments, a response panel is created by dividing each discrete cell population into a plurality of sample and contacting the samples with at least one modulator and determining an activation level of at least one activatable element in the samples. In some embodiments, each discrete cell population is contacted with a different modulator in separate cultures. In some embodiments, each discrete cell population is contacted with the same modulator in the same or separate cultures. In some embodiments, some discrete cell populations are contacted with the same modulator in the same or separate cultures, while other cell populations are contacted with a different modulator. For example, if the different discrete populations being analyzed are naive CD4 T cells, memory CD4 T cells, naive CD8 T cells and memory CD8 T cells, naive CD4 and memory CD4 can be contacted with the same first modulator in the same culture, while naive CD8 T cells and memory CD8 T cells are contacted with a second and third modulator, respectively, in separate cultures. The different discrete cells populations can be analyzed for the same activatable element or a different activatable element. The different discrete cells populations can be analyzed simultaneously or sequentially.

In some embodiments, the activatable element analyzed in each discrete cell population is different. In some embodiments, the activatable element analyzed in each discrete cell population is the same. In some embodiments, a plurality of activatable elements are analyzed in the discrete cell populations, where the activatable elements can be the same or different among the different discrete cell populations. In some embodiments, the number of activatable elements analyzed in each cell population is different. For example, in some embodiments only one activatable element is analyzed in one cell population, while a plurality (e.g. two or more) of activatable elements are analyzed in the other cell populations. When a plurality of activatable elements is analyzed in a discrete cell population, the activatable elements can be analyzed sequentially or simultaneously.

In some embodiments, the methods of the invention provide methods for generating activation state data for different discrete populations of cells by exposing each discrete population of cells to a plurality of modulators (recited herein) in separate cultures, determining the presence or absence of an increase in activation level of an activatable element in the discrete cell population from each of the separate cultures and classifying the discrete cell population based on the presence or absence of the increase in the activation of the activatable element from each of the separate culture. In some embodiments, activation state data is used to characterize multiple pathways in each of the population of cells. The activation state data of the different populations of cells can be used to determine the status of an individual or the state a cellular network.

The status of an individual or of a cellular network can be used in selecting a method of treatment. Example of methods of treatments include, but are not limited to chemotherapy, biological therapy, radiation therapy, bone marrow transplantation, Peripheral stem cell transplantation, umbilical cord blood transplantation, autologous stem cell transplantation, allogeneic stem cell transplantation, syngeneic stem cell transplantation, surgery, induction therapy, maintenance therapy, watchful waiting, and other therapy.

In addition to activation levels of activatable elements, expression levels of intracellular or extracellular biomolecules, e.g., proteins may be used alone or in combination with activation states of activatable elements to determine the status of an individual or a cellular network. Further, additional cellular elements, e.g., biomolecules or molecular complexes such as RNA, DNA, carbohydrates, metabolites, and the like, may be used in conjunction with activatable states or expression levels in the analysis of different discrete population of cells encompassed here. In some embodiments, expression markers are also measured in the different discrete cell populations. In some embodiments, expression markers or drug transporters, such as CD34, CD33, CD45, HLADR, CD11B FLT3 Ligand, c-KIT, ABCG2, MDR1, BCRP, MRP1, LRP, and others noted below, can also be used in the methods described herein. The expression markers may be detected using many different techniques, for example using nodes from flow cytometry data. Other common techniques employ expression arrays (commercially available from Affymetrix, Santa Clara Calif.), taqman (commercially available from ABI, Foster City Calif.), SAGE (commercially available from Genzyme, Cambridge Mass.), sequencing techniques (see the commercial products from Helicos, 454, US Genomics, and ABI) and other commonly know assays. See Golub et al., Science 286: 531-537 (1999). In some embodiments, the expression markers include epitope-based markers, RNA, mRNA, siRNA, or metabolomic markers.

In some embodiments, the invention provides methods to carry out multiparameter flow cytometry for monitoring phospho-protein responses to various factors in different discrete cell populations. Phospho-protein members of signaling cascades and the kinases and phosphatases that interact with them are required to initiate and regulate proliferative signals in cells. Flow cytometry is useful in a clinical setting, since relatively small sample sizes, as few as 10,000 cells, can produce a considerable amount of statistically tractable multidimensional signaling data. (See U.S. Pat. Nos. 7,381,535 and 7,393,656. See also Krutzik et al, 2004).

In the determination of a characteristic such as a prognostic or disease status of an individual, other factors can be considered. Any factor that gives additional predictive or diagnostic power to the analyses of different discrete cell populations described herein may be used. Such factors are well-known in the art. These include an individual's gender; race; current age; age at the time of disease presentation; age at the time of treatment; clinical stage of disease; genetic results, number of previous therapies; type of previous therapies; response to previous therapy or therapies; time from last treatment; blood cell count; bone marrow reserves; and performance status, patient's past medical history, family history of any medical problems, patient's social history, as well as any current medical history termed “review of systems”, and physical exam findings. Other factors are more specific to the specific condition being evaluated, e.g., percentage of blasts in bone marrow as an indicator of certain leukemias. Such factors are well-known in the art for particular diseases and conditions. Examples of tests that can be performed together with the methods described herein include, but are not limited to, immunophenotyping, morphology, conventional cytogenetics, molecular cytogenetics, molecular genetics and HLA typing.

### Modulators

In some embodiments, the methods and composition utilize a modulator. A modulator can be an activator, a therapeutic compound, an inhibitor or a compound capable of impacting a cellular pathway. Modulators can also take the form of environmental cues and inputs. Modulators can be uncharacterized or characterized as known compounds. A modulator can be a biological specimen or sample of a cellular or physiological environment from an individual, which may be a heterogeneous sample without complete chemical or biological characterization. Collection of the modulator specimen may occur directly from the individual, or be obtained indirectly. An illustrative example would be to remove a cellular sample from the individual, and then culture that sample to obtain modulators. A modulator can be present inside the individual, e.g. a chemical in a physiological environment inside the individual.

Modulation can be performed in a variety of environments. In some embodiments, cells comprising discrete cell populations are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells comprising discrete cell populations are exposed to at least 2, 3, 4, 5, 6, 7, 8, 9, or 10 modulators. See U.S. Patent Application 61/048,657 which is incorporated by reference. In some embodiments, discrete cell populations are exposed to a modulator while they are still inside the individual. For example, the individual has been exposed to chemical that is present in a physiological environment and as a result discrete cell populations have been exposed to that chemical.

In some embodiments, cells comprising discrete cells populations are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments the modulator is selected from the group consisting of growth factors, cytokines, adhesion molecules, drugs, hormones, small molecules, polynucleotides, antibodies, natural compounds, lactones, chemotherapeutic agents, immune modulators, carbohydrates, proteases, ions, reactive oxygen species, peptides, and protein fragments, either alone or in the context of cells, cells themselves, viruses, and biological and non-biological complexes (e.g. beads, plates, viral envelopes, antigen presentation molecules such as major histocompatibility complex). In some embodiments, the modulator is a physical stimuli such as heat, cold, UV radiation, and radiation. Examples of modulators, include but are not limited to SDF-1α, IFN-α, IFN-γ, IL-10, IL-6, IL-27, G-CSF, FLT-3L, IGF-1, M-CSF, SCF, PMA, Thapsigargin, H₂O₂, etoposide, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L.

In some embodiments, the modulator is an activator. In some embodiments the modulator is an inhibitor. In some embodiments, cells are exposed to one or more modulator. In some embodiments, cells comprising discrete cell populations are exposed to at least 2, 3, 4, 5, 6, 7, 8, 9, or 10 modulators. In some embodiments, cells comprising discrete cell populations are exposed to at least two modulators, wherein one modulator is an activator and one modulator is an inhibitor. In some embodiments, cells comprising discrete cell populations are exposed to at least 2, 3, 4, 5, 6, 7, 8, 9, or 10 modulators, where at least one of the modulators is an inhibitor. In some embodiments, the different discrete cell populations are exposed to the same modulators. In some embodiments, the different discrete cell populations are exposed to different modulators. For example, in some embodiments, the different discrete cell populations are exposed to the one or more modulators, where the one or more modulators are the same between the different discrete cell populations. In other embodiments, the different discrete cell populations are exposed to the one or more modulators, where the one or more modulators are different between the different discrete cell populations.

In some embodiments, the cross-linker is a molecular binding entity. In some embodiments, the molecular binding entity is a monovalent, bivalent, or multivalent is made more multivalent by attachment to a solid surface or tethered on a nanoparticle surface to increase the local valency of the epitope binding domain.

In some embodiments, the inhibitor is an inhibitor of a cellular factor or a plurality of factors that participates in a cellular pathway (e.g. signaling cascade) in the cell. In some embodiments, the inhibitor is a phosphatase inhibitor. Examples of phosphatase inhibitors include, but are not limited to H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenylarsine oxide, Pyrrolidine Dithiocarbamate, and Aluminium fluoride. In some embodiments, the phosphatase inhibitor is H₂O₂.

In some embodiments, the activation level of an activatable element in a discrete cell population is determined by contacting the discrete cell population with at least 2, 3, 4, 5, 6, 7, 8, 9, or 10 modulators. In some embodiments, the activation level of an activatable element in a discrete cell population is determined by contacting the discrete cell population with at least 2, 3, 4, 5, 6, 7, 8, 9, or 10 modulators where at least one of the modulators is an inhibitor. In some embodiments, the activation level of an activatable element in a discrete cell population is determined by contacting the discrete cell population with an inhibitor and a modulator, where the modulator can be an inhibitor or an activator. In some embodiments, the activation level of an activatable element in a discrete cell population is determined by contacting the discrete cell population with an inhibitor and an activator. In some embodiments, the activation level of an activatable element in a discrete cell population is determined by contacting the discrete cell population with two or more modulators. In some embodiments, the activation level of the same activatable element(s) is determined in different discrete cell populations. In some embodiments, the activation level of a different activatable element(s) is determined in different discrete cell populations. For example, in some embodiments, the activation level of the same activatable element(s) is determined in different discrete cell populations when the different discrete cells populations are exposed to one or more modulators, where the one or more modulators are the same between the different discrete cell populations. In some embodiments, the activation level of the same activatable element(s) is determined in different discrete cell populations when the different discrete cells populations are exposed to one or more modulators, where the one or more modulators are different between the different discrete cell populations. In some embodiments, the activation level of different activatable element(s) is determined in different discrete cell populations when the different discrete cells populations are exposed to one or more modulators, where the one or more modulators are the same between the different discrete cell populations. In some embodiments, the activation level of different activatable element(s) is determined in different discrete cell populations when the different discrete cells populations are exposed to one or more modulators, where the one or more modulators are different between the different discrete cell populations.

In some embodiments, the activation state a discrete cell population is determined by measuring the activation level of an activatable element when the population of cells is exposed to one or more modulators. The population of cells can be divided into a plurality of samples, and the activation state of the discrete cell population is determined by measuring the activation level of at least one activatable element in the samples after the samples have been exposed to one or more modulators. In some embodiments, the activation state different discrete cell populations are determined by measuring the activation level of an activatable element in each population of cells when each of the populations of cells is exposed to a modulator. The different populations of cells can be exposed to the same or different modulators. In some embodiments, the modulators include H₂O₂, PMA, SDF1 α, CD40L, IGF-1, IL-7, IL-6, IL-10, IL-27, IL-4, IL-2, IL-3, thapsigardin and/or a combination thereof. For instance a population of cells can be exposed to one or more, all or a combination of the following combination of modulators: H₂O₂; PMA; SDF1α; CD40L; IGF-1; IL-7; IL-6; IL-10; IL-27; IL-4; IL-2; IL-3; thapsigardin. In some embodiments, the physiological status of different cell discrete populations is used to determine the status of an individual as described herein.

### Activatable Elements

The methods and compositions of the invention may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously).

The activation state of an individual activatable element is either in the on or off state. As an illustrative example, and without intending to be limited to any theory, an individual phosphorylatable site on a protein will either be phosphorylated and then be in the “on” state or it will not be phosphorylated and hence, it will be in the “off” state. See Blume-Jensen and Hunter, Nature, vol 411, 17 May 2001, p 355-365. The terms “on” and “off,” when applied to an activatable element that is a part of a cellular constituent, are used here to describe the state of the activatable element (e.g., phosphorylated is “on” and non-phosphorylated is “off”), and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is typically measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis determining the activation levels of one or more activatable elements in cells may use the distribution of activation levels for one or more specific activatable elements which will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to determine the activation state data of a cell in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may not contain activatable elements, of one or more cells in a discrete population of cells may be used to determine the activation state data of the discrete cell population.

In some embodiments, the basis determining the activation state data of a discrete cell population may use the position of a cell in a contour or density plot. The contour or density plot represents the number of cells that share a characteristic such as the activation level of activatable proteins in response to a modulator. For example, when referring to activation levels of activatable elements in response to one or more modulators, normal individuals and patients with a condition might show populations with increased activation levels in response to the one or more modulators. However, the number of cells that have a specific activation level (e.g. specific amount of an activatable element) might be different between normal individuals and patients with a condition. Thus, the activation state data of a cell can be determined according to its location within a given region in the contour or density plot.

In addition to activation levels of intracellular activatable elements, expression levels of intracellular or extracellular biomolecules, e.g., proteins may be used alone or in combination with activation states of activatable elements to determine the activation state data of a population of cells. Further, additional cellular elements, e.g., biomolecules or molecular complexes such as RNA, DNA, carbohydrates, metabolites, and the like, may be used in conjunction with activatable states, expression levels or any combination of activatable states and expression levels in the determination of the physiological status of a population of cells encompassed here.

In some embodiments, other characteristics that affect the status of a cellular constituent may also be used to determine the activation state data of a discrete cell population. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to determine the activation state data of a discrete cell population, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, myeloid lineage cells can be further subdivided based on the expression of cell surface markers such as CD14, CD15, or CD33, CD34 and CD45.

Alternatively, populations of cells can be aggregated based upon shared characteristics that may include inclusion in one or more additional cell populations or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing characteristics.

In some embodiments, the activation state data of one or more cells is determined by examining and profiling the activation level of one or more activatable elements in a cellular pathway. In some embodiments, the activation levels of one or more activatable elements of a cell from a first discrete cell population and the activation levels of one or more activatable elements of cell from a second discrete cell population are correlated with a condition. In some embodiments, the first discrete cell population and second discrete cell population are hematopoietic cell populations. In some embodiments, the activation levels of one or more activatable elements of a cell from a first discrete cell population of hematopoietic cells and the activation levels of one or more activatable elements of cell from a second discrete cell population of hematopoietic cells are correlated with a neoplastic, autoimmune or hematopoietic condition as described herein. Examples of different discrete populations of hematopoietic cells include, but are not limited to, pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells.

In some embodiments, the activation level of one or more activatable elements in single cells in the sample is determined Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. The term “plurality” as used herein refers to two or more. In some embodiments, at least about 2, 3, 4, 5, 6, 7, 8, 9, 10, or more than 10 intracellular activatable elements are determined

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

In some embodiments, the activatable element is a protein. Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886, 61/048,920 and Shulz et al, Current Protocols in Immunology 2007, 7:8.17.1-20.

In some embodiments, the protein that may be activated is selected from the group consisting of HER receptors, PDGF receptors, FLT3 receptor, Kit receptor, FGF receptors, Eph receptors, Trk receptors, IGF receptors, Insulin receptor, Met receptor, Ret, VEGF receptors, erythropoetin receptor, thromobopoetin receptor, CD114, CD116, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pinl prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOX(O)STAT1, STAT 3, STAT 4, STAT 5, STAT 6, p53, WT-1, HMGA, pS6, 4EPB-1, eIF4E-binding protein, RNA polymerase, initiation factors, elongation factors.

In some embodiments of the invention, the methods described herein are employed to determine the activation level of an activatable element, e.g., in a cellular pathway. Methods and compositions are provided for the determination of the activation state data of a cell according to the activation level of an activatable element in a cellular pathway. Methods and compositions are provided for the determination of the activation state data of a cell in a first discrete cell population and a cell in a second discrete cell population according to the activation level of an activatable element in a cellular pathway in each cell. The cells can be a hematopoietic cell and examples are shown herein.

In some embodiments, the determination of the activation data of cells in different discrete cell populations according to activation level of an activatable element, e.g., in a cellular pathway, comprises classifying at least one of the cells as a cell that is correlated with a clinical outcome. Examples of clinical outcomes, staging, as well as patient responses are also shown herein.

(a) Signaling Pathways

In some embodiments, the methods of the invention are employed to determine the activation level of an activatable element in a signaling pathway. In some embodiments, the activation state data of a cell is determined, as described herein, according to the activation level of one or more activatable elements in one or more signaling pathways. Signaling pathways and their members have been extensively described. See (Hunter T. Cell Jan. 7, 2000; 100(1): 13-27; Weinberg, 2007; and Blume-Jensen and Hunter, Nature, vol 411, 17 May 2001, p 355-365 cited above). Exemplary signaling pathways include the following pathways and their members: the JAK-STAT pathway including JAKs, STATs 2, 3 4 and 5, the FLT3L signaling pathway, the MAP kinase pathway including Ras, Raf, MEK, ERK and elk; the PI3K/Akt pathway including PI-3-kinase, PDK1, Akt and Bad; the NF-KB pathway including IKKs, IkB and NF-KB and the Wnt pathway including frizzled receptors, beta-catenin, APC and other co-factors and TCF (see Cell Signaling Technology, Inc. 2002 Catalog pages 231-279 and Hunter T., supra.). In some embodiments of the invention, the correlated activatable elements being assayed (or the signaling proteins being examined) are members of the MAP kinase, Akt, NFkB, WNT, STAT and/or PKC signaling pathways.

In some embodiments, the methods of the invention are employed to determine the activation level of a signaling protein in a signaling pathway known in the art including those described herein. Exemplary types of signaling proteins within the scope of the present invention include, but are not limited to, kinases, kinase substrates (i.e. phosphorylated substrates), phosphatases, phosphatase substrates, binding proteins (such as 14-3-3), receptor ligands and receptors (cell surface receptor tyrosine kinases and nuclear receptors)). Kinases and protein binding domains, for example, have been well described (see, e.g., Cell Signaling Technology, Inc., 2002 Catalogue “The Human Protein Kinases” and “Protein Interaction Domains” pgs. 254-279).

Exemplary signaling proteins include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1, 2, 3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pinl prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, and elongation factors.

In some embodiments the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCyi, PLCy 2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKCα, PKC β, PKCθ, PKCδ, CAMK, Elk, AFT, Myc, Egr-1, NFAT, ATF-2, Mdm2, p53, DNA-PK, Chk1, Chk2, ATM, ATR, βcatenin, CrkL, GSK3α, GSK3β, and FOXO.

In some embodiments of the invention, the methods described herein are employed to determine the activation level of an activatable element in a signaling pathway. See U.S. Ser. Nos. 61/048,886 and 61/048,920 which are incorporated. Methods and compositions are provided for the determination of an activation state data of a cell according to the status of an activatable element in a signaling pathway. Methods and compositions are provided for the determination of a physiological status of cells in different populations of cells according to the status of an activatable element in a signaling pathway. The cells can be hematopoietic cells. Examples of hematopoietic cells are shown herein.

In some embodiments, the determination of an activation state data of cells in different populations of cells according to the activation level of an activatable element in a signaling pathway comprises classifying the cell populations as cells that are correlated with a clinical outcome. Examples of clinical outcome, staging, patient responses and classifications are shown above.

### Binding Element

In some embodiments of the invention, the activation level of an activatable element is determined. One embodiment makes this determination by contacting a cell from a cell population with a binding element that is specific for an activation state of the activatable element. The term “Binding element” includes any molecule, e.g., peptide, nucleic acid, small organic molecule which is capable of detecting an activation state of an activatable element over another activation state of the activatable element. Binding elements and labels for binding elements are shown in U.S. Ser. No./048,886; 61/048,920 and 61/048,657.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids for the purposes of the invention. The side chains may be in either the (R) or the (S) configuration. In some embodiments, the amino acids are in the S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. 5218: U138 Part 2 Aug. 22, 1999, both of which are expressly incorporated by reference herein.

Methods of the present invention may be used to detect any particular activatable element in a sample that is antigenically detectable and antigenically distinguishable from other activatable element which is present in the sample. For example, the activation state-specific antibodies of the present invention can be used in the present methods to identify distinct signaling cascades of a subset or subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. In some embodiments, the expression and phosphorylation of one or more polypeptides that are cellular components of a cellular pathway are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody.

The term “antibody” includes full length antibodies and antibody fragments, and may refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and posses other variations. See U.S. Ser. Nos. 61/048,886; 61/048,920 and 61/048,657 for more information about antibodies as binding elements.

Activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

The antigenicity of an activated isoform of an activatable element is distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of moieties to an element, such as phosphate moieties, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered around the active site of the element, although as is known in the art, conformational changes in one area of an element may cause alterations in different areas of the element as well.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoetin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pinl prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. In some embodiments, the protein is S6.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope may be used to produce such an antibody fragment by recombinant means well known in the art.

In alternative embodiments of the instant invention, aromatic amino acids of protein binding elements may be replaced with other molecules. See U.S. Ser. Nos. 61/048,886; 61/048,920 and 61/048,657.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55, each incorporated herein by reference). Further, fluorophores can be attached to such antibodies for use in the methods of the present invention.

A variety of recognitions structures are known in the art (e.g., Cochran et al., J. Am. Chem. Soc. (2001) 123:625-32; Boer et al., Blood (2002) 100:467-73, each expressly incorporated herein by reference)) and can be produced using methods known in the art (see e.g., Boer et al., Blood (2002) 100:467-73; Gualillo et al., Mol. Cell. Endocrinol. (2002) 190:83-9, each expressly incorporated herein by reference)), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In a some embodiment, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

In some embodiment the binding element is a small organic compound. Binding elements can be synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used in the present invention.

In some embodiments the binding element is a carbohydrate. As used herein the term carbohydrate is meant to include any compound with the general formula (CH₂0)ₙ. Examples of carbohydrates are di-, tri- and oligosaccharides, as well polysaccharides such as glycogen, cellulose, and starches.

In some embodiments the binding element is a lipid. As used herein the term lipid herein is meant to include any water insoluble organic molecule that is soluble in nonpolar organic solvents. Examples of lipids are steroids, such as cholesterol, and phospholipids such as sphingomeylin.

In some embodiments, the binding elements are used to isolated the activatable elements prior to its detection, e.g. using mass spectrometry.

Examples of activatable elements, activation states and methods of determining the activation level of activatable elements are described in US publication number 20060073474 entitled “Methods and compositions for detecting the activation state of multiple proteins in single cells” and US publication number 20050112700 entitled “Methods and compositions for risk stratification” the content of which are incorporate here by reference.

(a) Labels

The methods and compositions of the instant invention provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. Binding elements and labels for binding elements are shown in U.S. Ser. No./048,886; 61/048,920 and 61/048,657.

A compound can be directly or indirectly conjugated to a label which provides a detectable signal, e.g. radioisotopes, fluorescers, enzymes, antibodies, particles such as magnetic particles, chemiluminescers, molecules that can be detected by mass spec, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments of the invention, these labels may be conjugated to the binding elements.

In some embodiments, one or more binding elements are uniquely labeled. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Labels include optical labels such as fluorescent dyes or moieties. Fluorophores can be either “small molecule” fluors, or proteinaceous fluors (e.g. green fluorescent proteins and all variants thereof).

In some embodiments, activation state-specific antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies can be used alone or they can be employed in conjunction with organic fluorochrome—conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 March; 62(3):188-195.

Alternatively, detection systems based on FRET, discussed in detail below, may be used. FRET finds use in the instant invention, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

The methods and composition of the present invention may also make use of label enzymes. By label enzyme is meant an enzyme that may be reacted in the presence of a label enzyme substrate that produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By radioisotope is meant any radioactive molecule. Suitable radioisotopes for use in the invention include, but are not limited to ¹⁴C, ³H, ³²P, ³³P, ³⁵S, ¹²⁵I and ¹³¹I. The use of radioisotopes as labels is well known in the art.

As mentioned, labels may be indirectly detected, that is, the tag is a partner of a binding pair. By “partner of a binding pair” is meant one of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255: 192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. As will be appreciated by those in the art, binding pair partners may be used in applications other than for labeling, as is described herein.

As will be appreciated by those in the art, a partner of one binding pair may also be a partner of another binding pair. For example, an antigen (first moiety) may bind to a first antibody (second moiety) that may, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. Attaching a label to a tag that is a partner of a binding pair, as just described, is referred to herein as “indirect labeling”.

By “surface substrate binding molecule” or “attachment tag” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

In some embodiments, the activatable elements are labeled by incorporating a label as describing herein within the activatable element. For example, an activatable element can be labeled in a cell by culturing the cell with amino acids comprising radioisotopes. The labeled activatable element can be measured using, for example, mass spectrometry.

### Alternative Activation State Indicators

An alternative activation state indicator useful with the instant invention is one that allows for the detection of activation by indicating the result of such activation. For example, phosphorylation of a substrate can be used to detect the activation of the kinase responsible for phosphorylating that substrate. Similarly, cleavage of a substrate can be used as an indicator of the activation of a protease responsible for such cleavage. Methods are well known in the art that allow coupling of such indications to detectable signals, such as the labels and tags described above in connection with binding elements. For example, cleavage of a substrate can result in the removal of a quenching moiety and thus allowing for a detectable signal being produced from a previously quenched label. In addition, binding elements can be used in the isolation of labeled activatable elements which can then be detected using techniques known in the art such as mass spectrometry.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detect and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See U.S. patent Ser. No. 10/898,734 and Shulz et al., Current Protocols in Immunology, 2007, 78:8.17.1-20 which are incorporated by reference in their entireties.

In some embodiments, the present invention provides methods for determining the activation level on an activatable element for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Binding elements can also be used to isolate activatable elements which can then be analyzed by methods known in the art. Alternatively, non-binding elements systems as described above can be used in any system described herein.

When using fluorescent labeled components in the methods and compositions of the present invention, it will recognize that different types of fluorescent monitoring systems, e.g., Cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells that may used as a modulator or as a population of reference cells. In some embodiments, the modulator or reference cells are first contacted with fluorescent-labeled binding elements (e.g. antibodies) directed against specific elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic filed while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation level of at least one activatable element. In some embodiments, cells are analyzed on the basis of the activation level of at least two activatable elements.

In some embodiments, a multiplicity of activatable element activation-state antibodies is used to simultaneously determine the activation level of a multiplicity of elements.

In some embodiment, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

As will be appreciated, these methods provide for the identification of distinct signaling cascades for both artificial and stimulatory conditions in cell populations, such a peripheral blood mononuclear cells, or naive and memory lymphocytes.

When necessary, cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200C; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, 2007 March; 62(3):188-195.).

As will be appreciated by one of skill in the art, the instant methods and compositions find use in a variety of other assay formats in addition to flow cytometry analysis. For example, a chip analogous to a DNA chip can be used in the methods of the present invention. Arrayers and methods for spotting nucleic acids on a chip in a prefigured array are known. In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell. See U.S. Pat. No. 5,744,934. In some embodiments, a microfluidic image cytometry is used (Sun et al. Cancer Res; 70(15) Aug. 1, 2010)

In some embodiments confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiments, the methods of the invention include the use of liquid handling components. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems. See U.S. Ser. No. 61/048,657 which is incorporated by reference in its entirety.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader. See U.S. Ser. No. 61/048,657.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory. See U.S. Ser. No. 61/048,657 which is incorporated by reference in its entirety.

These robotic fluid handling systems can utilize any number of different reagents, including buffers, reagents, samples, washes, assay components such as label probes, etc.

Any of the steps above can be performed by a computer program product that comprises a computer executable logic that is recorded on a computer readable medium. For example, the computer program can execute some or all of the following functions: (i) exposing different population of cells to one or more modulators, (ii) exposing different population of cells to one or more binding elements, (iii) detecting the activation levels of one or more activatable elements, and (iv) making a diagnosis or prognosis based on the activation level of one or more activatable elements in the different populations.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of determining the status of an individual by accessing data that reflects the activation level of one or more activatable elements in the reference population of cells.

### Conditions

The methods of the invention are applicable to any condition in an individual involving, indicated by, and/or arising from, in whole or in part, altered physiological status in cells. The term “physiological status” includes mechanical, physical, and biochemical functions in a cell. In some embodiments, the physiological status of a cell is determined by measuring characteristics of at least one cellular component of a cellular pathway in cells from different populations (e.g. different cell networks). Cellular pathways are well known in the art. In some embodiments the cellular pathway is a signaling pathway. Signaling pathways are also well known in the art (see, e.g., Hunter T., Cell 100(1): 113-27 (2000); Cell Signaling Technology, Inc., 2002 Catalogue, Pathway Diagrams pgs. 232-253; Weinberg, Chapter 6, The biology of Cancer, 2007; and Blume-Jensen and Hunter, Nature, vol 411, 17 May 2001, p 355-365). A condition involving or characterized by altered physiological status may be readily identified, for example, by determining the state of one or more activatable elements in cells from different populations, as taught herein.

In certain embodiments of the invention, the condition is a neoplastic, immunologic or hematopoietic condition. In some embodiments, the neoplastic, immunologic or hematopoietic condition is selected from the group consisting of solid tumors such as head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma, sepsis, allergic diseases and disorders that include but are not limited to allergic rhinitis, allergic conjunctivitis, allergic asthma, atopic eczema, atopic dermatitis, and food allergy, immunodeficiencies including but not limited to severe combined immunodeficiency (SCID), hypereosiniphic syndrome, chronic granulomatous disease, leukocyte adhesion deficiency I and II, hyper IgE syndrome, Chediak Higashi, neutrophilias, neutropenias, aplasias, agammaglobulinemia, hyper-IgM syndromes, DiGeorge/Velocardial-facial syndromes and Interferon gamma-TH1 pathway defects, autoimmune and immune dysregulation disorders that include but are not limited to rheumatoid arthritis, diabetes, systemic lupus erythematosus, Graves' disease, Graves ophthalmopathy, Crohn's disease, multiple sclerosis, psoriasis, systemic sclerosis, goiter and struma lymphomatosa (Hashimoto's thyroiditis, lymphadenoid goiter), alopecia aerata, autoimmune myocarditis, lichen sclerosis, autoimmune uveitis, Addison's disease, atrophic gastritis, myasthenia gravis, idiopathic thrombocytopenic purpura, hemolytic anemia, primary biliary cirrhosis, Wegener's granulomatosis, polyarteritis nodosa, and inflammatory bowel disease, allograft rejection and tissue destructive from allergic reactions to infectious microorganisms or to environmental antigens, and hematopoietic conditions that include but are not limited to Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibroses, or atypical immune lymphoproliferations. In some embodiments, the neoplastic or hematopoietic condition is non-B lineage derived, such as Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, or non-B atypical immune lymphoproliferations, Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia.

In some embodiments, the neoplastic or hematopoietic condition is non-B lineage derived. Examples of non-B lineage derived neoplastic or hematopoietic condition include, but are not limited to, Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B atypical immune lymphoproliferations.

In some embodiments, the neoplastic or hematopoietic condition is a B-Cell or B cell lineage derived disorder. Examples of B-Cell or B cell lineage derived neoplastic or hematopoietic condition include but are not limited to Chronic Lymphocytic Leukemia (CLL), B lymphocyte lineage leukemia, B lymphocyte lineage lymphoma, Multiple Myeloma, and plasma cell disorders, including amyloidosis and Waldenstrom's macroglobulinemia.

Other conditions within the scope of the present invention include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998;95(9):5246-50), increased IGF-1 expression in prostate cancer (Schaefer et al., Science Oct. 9, 1998, 282: 199a), EGFR overexpression and resulting ERK activation in glioma cancer (Thomas C Y. Int J Cancer Mar. 10, 2003;104(1):19-27), expression of HER2 in breast cancers (Menard et al. Oncogene. Sep 29 2003, 22(42):6570-8), and APC mutation and activated Wnt signaling in colon cancer (Bienz M. Curr Opin Genet Dev 1999 October, 9(5):595-603).

Diseases other than cancer involving altered physiological status are also encompassed by the present invention. For example, it has been shown that diabetes involves underlying signaling changes, namely resistance to insulin and failure to activate downstream signaling through IRS (Burks D J, White M F. Diabetes 2001 February; 50 Suppl 1:S140-5). Similarly, cardiovascular disease has been shown to involve hypertrophy of the cardiac cells involving multiple pathways such as the PKC family (Malhotra A. Mol Cell Biochem 2001 September; 225 (1-):97-107). Inflammatory diseases, such as rheumatoid arthritis, are known to involve the chemokine receptors and disrupted downstream signaling (D'Ambrosio D J Immunol Methods 2003 February; 273 (1-2):3-13) and are also encompassed herein. Transplant rejection, infections (e.g. viral or bacterial), and vaccines state responses are also encompassed in the invention. Examples of vaccine state responses that can be measured by the methods described herein are described in U.S. provisional application No. 61/327,347 incorporate by reference herein in its entirety for all purposes. The invention is not limited to diseases presently known to involve altered cellular function, but includes diseases subsequently shown to involve physiological alterations or anomalies.

### Kits

In some embodiments the invention provides kits. Kits provided by the invention may comprise one or more of the state-specific binding elements described herein, such as phospho-specific antibodies. A kit may also include other reagents that are useful in the invention, such as modulators, fixatives, containers, plates, buffers, therapeutic agents, instructions, and the like.

In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCγ1, PLCγ2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25,A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, \tilde{β}catenin, CrkL, GSK3α, GSK3β, and FOXO. In some embodiments, the kit comprises one or more of the phospho-specific antibodies specific for the proteins selected from the group consisting of Erk, Syk, Zap70, Lck, Btk, BLNK, Cbl, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP70, Btk, BLNK, Lck, PLCy, PLCy 2, STAT1, STAT 3, STAT 4, STAT 5, STAT 6, CREB, Lyn, p-S6, Cbl, NF-κB, GSK3β, CARMA/Bcl10 and Tcl-1.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### EXAMPLES

### Example 1

### Analysis of AML Patients

Patient samples: Sets of fresh or cryopreserved samples from patients can be analyzed. The sets can consist of peripheral blood mononuclear cell (PBMC) samples or bone marrow mononuclear cell (BMMC) samples derived from the blood of AML patients. All patients will be asked for consent for the collection and use of their samples for institutional review board (IRB)-approved research purposes. All clinical data is de-identified in compliance with Health Insurance Portability and Accountability Act (HIPAA) regulations. Sample inclusion criteria can require collection at a time point prior to initiation of induction chemotherapy, AML classification by the French-American-British (FAB) criteria as M0 through M7 (excluding M3), and availability of appropriate clinical annotations (e.g., disease response after one or two cycles of induction chemotherapy). Induction chemotherapy can consist of at least one cycle of standard cytarabine-based induction therapy (i.e., daunorubicin 60 mg/m2×3 days, cytarabine 100-200 mg/m2 continuous infusion×7 days); responses are measured after one cycle of induction therapy. Standard clinical and laboratory criteria can be used for defining complete responders (CR) in the patient samples. Leukemia samples obtained from patients who do not meet the criteria for CR or samples obtained from those who died during induction therapy are considered non-complete response (NR) for the primary analyses.

Cell network profiling assays: Cell network profiling assays involved measuring the expression of protein levels and their post-translational modification by phosphorylation in different populations of cells at baseline and after perturbation with various modulators. The populations that can be analyzed include myeloid leukemic cells, B cells, T cells, dendritic cells, monocytes, macrophages, neutrophils, eosinophils, and basophils. Other cells such as epithelial cells can also be analyzed.

A pathway “node” is defined as a combination of a specific proteomic readout in the presence or absence of a specific modulator. Levels of signaling proteins, as well as expression of cell surface markers (including cell lineage markers, membrane receptors and drug transporters), are detected by multiparameter flow cytometry using fluorochrome-conjugated antibodies to the target proteins. Multiple nodes (including surface receptors and transporters), using multiple modulators can be assessed in the two studies.

A minimum yield of 100,000 viable cells and 500 cells per gated sample in gate of interest can be used for each patient sample to be classified as evaluable.

Cyropreserved samples are thawed at 37° C., washed, and centrifuged in PBS, 10% FBS, and 2 mM EDTA. The cells are resuspended, filtered, and are washed in RPMI cell culture media, 1% FBS, then are stained with Live/Dead Fixable Aqua Viability Dye (Invitrogen, Carlsbad, Calif.) to distinguish non-viable cells. The viable cells are resuspended in RPMI, 1% FBS, aliquoted to 100,000 cells/condition, and are rested for 1-2 hours at 37° C. prior to cell-based functional assays or staining for phenotypic markers. Each condition can include 2 to 5 phenotypic markers (e.g., CD45, CD33), up to 3 intracellular stains, or up to 3 additional surface markers.

Cells are incubated with modulators, at 37° C. for 3-15 minutes, then fixed with 1.6% paraformaldehyde (final concentration) for 10 minutes at 37° C., pelleted, and permeabilized with 100% ice-cold methanol and stored at −20° C. For functional apoptosis assays, cells are incubated for 24 hours with cytotoxic drugs (i.e. Etoposide or Ara-C and daunorubicin), then re-stained with Live/Dead Fixable Aqua Viability Dye to distinguish non-viable cells before fixation and permeabilization, washed with FACS Buffer (PBS, 0.5% BSA, 0.05% NaN3), pelleted, and stained with fluorescent dye-conjugated antibodies (Becton Dickenson-Pharmingen, San Diego, Calif.) to both surface antigens (CD33, CD45) and the signaling protein targets.

Data acquisition and cytometry analysis: Data is acquired using FACS DIVA software on both LSR II and CANTO II Flow Cytometers (BD). For all analyses, dead cells and debris are excluded by FSC (forward scatter), SSC (side scatter), and Amine Aqua Viability Dye measurement. Leukemic cells are identified as cells that lacked the characteristics of mature lymphocytes (CD45++, CD33−), and that fit the CD45 and CD33 versus right-angle light-scatter characteristics consistent with myeloid leukemia cells. Other cell populations are identified using markers known in the art.

Statistical Analysis and Stratifying Node Selection

a) Metrics:

The median fluorescence intensity (MFI) is computed for each node from the intensity levels for the cells in the gate of interest. The MFI values are then used to compute a variety of metrics by comparing them to the various baseline or background values, i.e. the unstimulated condition, autofluorescence, and isotype control. The following metricscan be computed in these studies: (1) Basal MFI=log 2(MFIUnmodulated Stained)−log 2(MFIGated Unstained (Autofluoresence)), designed to measure the basal levels of a certain protein under unmodulated conditions; (2) Fold Change MFI=log 2(MFIModulated Stained)−log 2(MFIUnmodulated Stained), a measure of the change in the activation state of a protein under modulated conditions; (3) Total Phospho MFI=log 2(MFIModulated Stained)−log 2(MFIGated Unstained (Autofluorescence)), a measure of the total levels of a protein under modulated conditions; (4) Fold over Control MFI=log 2(MFIStain)−log 2(MFIControl), a measure of the levels of surface marker staining relative to control antibody staining; (5) Percent Cell Positivity=a measure of the frequency of cells that have surface markers staining at an intensity level greater than the 95th percentile for control antibody staining

An additional metric is designed to measure the levels of cellular apoptosis in response to cytotoxic drugs: (6) Quadrant=a measure of the percentage of cells expressing high levels of apoptosis molecules (e.g. cleaved PARP and low levels of p-Chk2)

A low signaling node is defined as a node having a fold change metric or total phosphoprotein signal equal to I log 2(Fold) I>0.15. However, it is not necessary to use this as an exclusion criterion in this study.

b) Reproducibility Analysis

Two or more cryopreserved vials or fresh samples for each evaluable patient sample are obtained. All the vials are processed separately to access the assay reproducibility. Pearson and Spearman rank correlations were computed for each node/metric combination between the two data sets.

c) Univariate Analysis

All cell population/node/metric combinations are analyzed and compared across samples for their ability to distinguish between CR and NR samples. For each cell population/node/metric combination student t-test and Wilcoxon test p-Values are computed. In addition, the area under the receiver operator characteristic (ROC) (Hanley and McNeil, Radiology, 1982, Hanley and McNeil, Radiology, 1983, Bewick, et al, Critical Care, 2004) curve is also computed to access the diagnostic accuracy of each node for a given metric. The sensitivity (proportion of patients for whom a CR is correctly identified) and specificity (proportion of patients for whom a NR is correctly identified) data are plotted as ROC curves. A random result would produce an AUC value of 0.5. A (bio)marker with 100% specificity and selectivity would result in an AUC of 1.0. The cell population/node/metric combinations are independently tested for differences between patient samples whose response to standard induction therapy was CR vs NR. No corrections are applied to the p-values to correct for multiple testing. Instead, simulations are performed by randomly permuting the clinical variable to estimate the number of cell population/node/metric combinations that might appear to be significant by chance. For each permutation, nine donors are randomly chosen (without replacement) and assigned to the CR category and the remaining are assigned to the NR category. By comparing each cell population/node/metric combination to the permuted clinical variable, the student t-test p-values are computed. This process is repeated. The results from these simulations are then used to estimate the number of cell population/node/metric combinations that are expected to be significant by chance at the various p-values and compared with the empirical p-values for the number of cell population/node/metric combinations that were found to be significant from the real data.

The statistical analyses can be performed with the statistical software package R, version 2.7.0

d) Correlations Between Node:

Correlations between all pairs of cell population/node/metric combinations are accessed by computing Pearson and Spearman rank correlation.

e) Combinations of Nodes

Nodes that can potentially complement each other in combination to improve the accuracy of prediction of response to therapy are also explored. With a small size of the data set, a straightforward “corner classifier” approach for picking combinations can be adopted. Combinations that seem promising are also tested for their stability via a bootstrapping approach described below.

The corners classifier is a rules-based algorithm for dividing subjects into two classes (in this case the dichotomized response to induction therapy) using one or more numeric variables (defined in our study as a node/metric combination). This method works by setting a threshold on each variable, and then combining the resulting intervals (e.g., X<10, or Y>50) with the conjunction (and) operator (reference). This creates a rectangular region that is expected to hold most members of the class previously identified as the target (in this study CR or NR samples). Threshold values are chosen by minimizing an error criterion based on the logit-transformed misclassification rate within each class. The method assumes only that the two classes (i.e. response or lack of response to induction therapy) tend to have different locations along the variables used, and is invariant under monotone transformations of those variables.

A bagging, also known as bootstrapped aggregation, is used i to internally cross-validate the results of the above statistical model. Bootstrap re-samples are drawn from the original data. Each classifier, i.e. combination of cell population/node/metric, is fit to the resample, and then used to predict the class membership of those patients who were excluded from the resample. After repeating the re-sampling operation sufficiently, each patient acquires a list of predicted class memberships based on classifiers that are fit using other patients. Each patient's list is reduced to the fraction of target class predictions; members of the target class should have fractions near 1, unlike members of the other class. The set of such fractions, along with the patient's true class membership, is used to create an ROC curve and to calculate its AUC.

### Example 2

### Analysis of Rheumatoid Arthritis patients

Patient samples: Sets of fresh or cryopreserved samples from patients can be analyzed. The sets can consist of cells samples derived from the lymph nodes, synovium and/or synovial fluid of rheumatoid patients. All patients will be asked for consent for the collection and use of their samples for institutional review board (IRB)-approved research purposes. All clinical data is de-identified in compliance with Health Insurance Portability and Accountability Act (HIPAA) regulations.

Sample inclusion criteria can include: (i) A diagnosis of rheumatoid arthritis by the 1987 ACR criteria, (ii) Definite bony erosions, (iii) Age of disease onset greater than 18 years. (iv) Patient does not have psoriasis, inflammatory bowel disease, or systemic lupus erythematosus.

Standard clinical and laboratory criteria can be used for defining RA patients that are able to respond to a treatment in the patient samples. RA samples obtained from patients who do not meet the criteria for patients that are able to respond are considered non-complete responders for the primary analyses. Examples of possible treatments include nonsteroidal antiinflammatory drugs (NSAIDs) such as Acetylsalicylate (aspirin), naproxen (Naprosyn), ibuprofen (Advil, Medipren, Motrin), and etodolac (Lodine); Corticosteroid; Hydroxychloroquine; Sulfasalazine (Azulfidine); Gold salts such as Gold thioglucose (Solganal), gold thiomalate (Myochrysine), and auranofin (Ridaura); D-penicillamine (Depen, Cuprimine); Immunosuppressive medicines such as methotrexate (Rheumatrex, Trexall), azathioprine (Imuran), cyclophosphamide (Cytoxan), chlorambucil (Leukeran), and cyclosporine (Sandimmune).

Populations of cells that can be analyzed using the methods described in Example 1 include B cells, T cells, dendritic cells, monocytes, macrophages, neutrophils, eosinophils, and basophils. Other cells such as mesechymal cells and epithelial cells can also be analyzed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 3

### Cellular and Intracellular Network Characterization of Cytokine JAK/STAT Signaling in Whole Blood Across Multiple Healthy Individuals: Defining “Normal”

Aberrant JAK/STAT signaling in hematopoietic cells has shown to be involved in certain hematological and immune diseases; thus, the regulation of JAK/STAT signaling is an important research area. Signaling pathway- and cell type-specific responses to various cytokines in the immune system signaling network can elicit a wide range of biological outcomes due to the combinatorial use of a limited set of kinases and STAT proteins. Although advances have been made in uncovering the intracellular mechanisms relating to cytokine signaling, the biological outcome may vary depending on composition and activation state of the cellular network. Single Cell Network Profiling (SCNP) by flow cytometry allows the interrogation of intracellular signaling networks within a heterogeneous cellular network, such as in unfractionated whole blood. We applied SCNP to investigate cytokine-induced JAK/STAT signaling in whole blood across healthy human donors (n=11) to 1) measure the relative contribution of signaling across multiple cell subsets; 2) measure the kinetics of signaling activation and resolution across cytokines and cell subsets; 3) measure the variation among donors in their overall signaling characteristics. Our aim was to better characterize “normal” cytokine responses across healthy individuals as a basis to eventually describe abnormal states.

Method: Whole blood from 11 healthy donors (20-65 yrs, 7 males, 4 females, 8 Caucasians, 2 Hispanics, 1 East Asian) was stimulated at 37° C. in 96-well plates with a low, medium, and high dose of GM-CSF, IFN-α, IL-27 and IL-6, each added separately, as described in Example 5. For each dose, a stimulation time course was run with 6 time points between 3 and 45 minutes. Each well had a final concentration of 90% whole blood. The SCNP assay was performed using a fluorophore-labeled antibody cocktail to simultaneously measure signaling in six distinct cell populations, including: neutrophils, CD20+B cells, CD3+CD4+ T cells, CD3+CD4−T cells (CD8 enriched), CD3−CD20− lymphocytes (NK cell enriched), and CD14+ monocytes. The median fluorescent intensity of phospho (p)-STAT1(Y701), p-STAT3(Y705), and p-STAT5(Y694) were measured in each defined cell population for each experimental condition.

Results: This SCNP assay was relatively high-throughput and provided high-content data, that equates to 19,000 gel lanes if attempted by Western analysis (11 donors×4 cytokines×4 concentrations×6 time points×6 cell subsets×3 p-readouts). In general, each cytokine demonstrated unique dose-dependent signaling characteristics (e.g., activation/termination kinetics, magnitude of response) for each cell type analyzed, and in some cases, the kinetics differed between p-STAT readouts within the same cell subset for the same cytokine. For instance, IL-6 induced signaling was only observed in CD4+ T cells and monocytes with peak p-STAT3 levels at 3 minutes followed by p-STAT1 and p-STATS at 10-15 minutes. In addition, signal resolution fell to baseline levels at 45 minutes in monocytes, while the CD4+ T cells showed sustained elevated signaling, suggesting a cell-type specific regulation. In contrast to IL-6, IFN-α, stimulation activated all 3 STAT proteins, peaking at 10 minutes with similar kinetics in all cell subsets. However, IFN-α signaling resolution was faster and almost complete at 45 minutes in monocytes, while in the all other subsets the signal was sustained. This efficient signal termination in monocytes was also observed with GM-CSF→p-STAT5, while neutrophils maintained persistent p-STATS levels. IL-27 induced p-STAT1 and p-STAT3 in T cell subsets, B cells, and monocytes with peak activation at 30 minutes. In general, signaling characteristics were remarkably uniform across the healthy donors. IL-6→p-STAT3 was particularly consistent across time points and ligand concentrations, while p-STAT1 and p-STATS showed more variation. More results are provided in Example 5.

Approaching cell signaling from the perspective of the cellular network under physiological conditions (whole blood) allows for a more comprehensive and clinically relevant view of the signaling state of complex tissues. As many JAK/STAT targeting small molecule compounds enter the clinic, this study provides an important reference point for comparison with signaling networks that have become altered either by the pathological disease state or by therapy.

### Example 4

### Single Cell Network Profiling (SCNP) of IFN-α Signaling Pathways in Peripheral Blood Mononuclear Cells from Healthy Donors: Implications for Disease Characterization, Treatment Selection, and Drug Discovery

The antiviral and antitumor effects of IFN-α, have been exploited for the treatment of viral infections such as hepatitis C(HCV) as well as for various malignancies, such as hairy cell leukemia and melanoma. However, widespread use of IFN-α for these and other indications is severely hampered by significant side effects which can have a major impact on patient quality of life. Thus, a greater understanding of intracellular signaling pathways regulated by IFN-α may guide in the selection of patients whose disease will have an optimal response with tolerable side effects to this cytokine. Specifically, the Signal Transducer and Activation of Transcription (Stat) transcription factors are known to play a critical role in transducing IFN-a mediated signals. Single cell network profiling (SCNP) is a multiparameter flow-cytometry based approach that can be used to simultaneously measure extracellular surface makers and intracellular signaling proteins in individual cells in response to externally added modulators. Here, we use SCNP to interrogate IFN-a signaling pathways in multiple cell subsets within peripheral blood mononuclear cells (PBMCs) from healthy donors.

This study was designed to apply SCNP to generate a map of IFN-α-mediated signaling responses, with emphasis on Stat proteins, in PBMCs from healthy donors. The data provides a reference for future studies using PBMCs from patient samples in which IFN-α-mediated signaling is aberrantly regulated.

Methods: IFN-α-mediated signaling responses were measured by SCNP in PBMC samples from 12 healthy donors. PBMCs were processed for flow cytometry by fixation and permeabilization followed by incubation with fluorochrome-conjugated antibodies that recognize extracellular lineage markers and intracellular signaling molecules. The levels of several phospho-proteins (p-Stat1, p-Stat3, p-Stat4, p-Stat5, p-Stat6, and p-p38) were measured in multiple cell populations (CD14+ monocytes, CD20+B cells, CD4+ CD3+ T cells, and CD4− CD3+ T cells) at 15 minutes, 1, 2 and 4 hours post IFN-α exposure as described in Example 6.

Results: The data revealed distinct phospho-protein activation patterns in different cell subsets within PBMCs in response to IFN-α exposure. For example, activation of p-Stat4 was detected in T cell subsets (both CD4+ and CD4− T cells), but not in monocytes or B cells. Such cell-type specific activation patterns likely play a key role in mediating specific functions within different cell types in response to IFN-α. Differences in the kinetics of activation by IFN-α for different phospho-proteins were also observed. The peak response for activation of p-Stat1, p-Stat3, and p-Stat5 was at 15 minutes in most of the cell types interrogated in this study, whereas for the activation of p-Stat4, p-Stat6, and p-p38 it was at 1 hr in the majority of cell types tested. The relationships between phospho-protein readouts in each cell subset were determined by calculating the Pearson correlation coefficients. For example, the activation of p-Stat1 and p-Stat5 at 15 minutes was positively correlated in both B cells and T cells. More results are provided in Example 6.

The activation of intracellular signaling proteins was measured with emphasis on Stat transcription factors in PBMC subsets from healthy donors. We have analyzed the relationships between the activation states of phospho-proteins in the IFN-α signaling network. Characterization of IFN-α signaling pathways in samples from healthy donors has provided a network map that can be used as a reference for identifying alterations in IFN-α signaling that are the consequence of disease and/or therapeutic intervention. Future studies using SCNP to characterize IFN-α signaling pathways in PBMCs from patients with diseases such as viral infections or cancer may enable the optimization of IFN-α dosing and the identification of patient stratification biomarkers as well as the discovery of novel therapeutic agents.

### Example 5

### Normal Cell Response to Erythropoietin(EPO) and Granulocyte Colony Stimulating Factor (G-CSF)

Normal cell signaling response to EPO and G-CSF was characterized through comparison to signaling response observed in samples from a subclass of patients with myelodysplatic syndrome (MDS) referred to herein as “low risk” patients. 15 samples of healthy BMMCs (from patients with no known diagnosis of disease) and 14 samples of BMMCs from patients who belonged to a subclass of patients with myelodysplastic syndrome were used to characterize normal cell response. The 14 samples of low risk patients were obtained from MD Anderson Cancer Center in Texas. The low risk patients were diagnosed as per standard of care at MD Anderson Cancer Center. The 15 samples of healthy BMMCs were obtained through Williamson Medical Center and from a commercial source (AllCells, Emeryville, Calif.). The samples obtained through Williamson Medical Center were collected with informed consent from patients undergoing surgeries such as knee or hip replacements.

Each of the normal and the low risk samples were separated in aliquots. The aliquots were treated with a 3 IU/ml concentration of Erythropoietin, a 50 ng/ml concentration of G-CSF and both a 3 IU/ml concentration of Erythropoietin and a 50 ng/ml concentration of G-CSF. Activation levels of pStat1, pStat3 and pStat5 were measured using flow cytometry at 15 minutes after treatment with the modulators. In addition to the Stat proteins measured, several other elements were measured in order to separate the cells into discrete populations according to cell type. These markers included CD45, CD34, CD71 and CD235ab. CD45 was used to segregate Lymphocytes, Myeloid(p1) cells and nRBCs. The nRBCs were further segregated into 4 distinct cell populations based on expression of CD71 and CD235ab: m1, m2, m3 and m4. These cell populations correspond to RBC maturity and are illustrated in FIG. 2.

Distinct signaling responses were observed in the different discrete cell populations. FIG. 2 illustrates the different activation levels of pStat1, pStat3 and pStat5 observed in EPO, G-CSF and EPO+G-CSF treated Lymphocytes, nRBC1 cells, Myeloid(p1) cells and stem cells. Activation levels observed in different samples from the normal and low risk populations are plotted as dots. As shown in FIG. 2, different cell discrete populations demonstrated different induced activation levels. Although this was true in both the healthy and the low risk patients, the different discrete cell populations exhibited a narrower range of induced activation levels in then normal samples than in the low risk samples. These observations accord with the common understanding that diseased cells exhibit a wider range of different signaling phenotypes than normal cells.

Additionally, cell differentiation in disease may be inhibited or stunted, causing cells to exhibit characteristics that are different from other cells of the same type.

### Example 6

### Normal Cell Response to Varying Concentrations of GM-CSF, IL-27, IFNα and IL-6

Kinetic response to varying concentrations of modulators was investigated in normal samples (i.e. samples from persons who have no diagnosis of disease). 11 normal samples were donated with informed consent by Nodality Inc. employees and processed at Nodality Inc. in South San Francisco, Calif. The samples were treated with 4 different modulators (GM-CSF, IL-27, IFNα and IL-6) at 4 different concentrations of the modulator and activation levels of pStat1, pStat3 and pStat5 were measured at different time points. Activation levels were measured at 3, 5, 10, 15, 30 and 45 minutes using flow cytometry-based single cell network profiling. The concentrations of the stimulators are tabulated below:

TABLE 1
Stimulator Concentrations

| | low | med | hi |
|---|---|---|---|
| GM-CSF | 0.1 ng/ml | 1 ng/ml | 10 ng/ml |
| IL-27 | 1 ng/ml | 10 ng/ml | 100 ng/ml |
| IFNa | 1000 IU | 4000 IU | 100000 IU |
| IL-6 | 1 ng/ml | 10 ng/ml | 100 ng/ml |

Activation levels of different cell surface markers were also profiled using single cell network profiling and used in conjunction with gating to segregate the cells into discrete cell populations. In the gating analysis, SSC-A and FSC-A were first used to segregate lymphocytes from non-lymphocytes. CD14 and CD4 were then used to segregate the non-lymphocytes into populations of neutrophils and CD 14+ cells (monoctyes). CD3 and CD20 were then used to segregate the lymphocytes into populations of CD20+ (B Cells), CD3+ (T Cells) and CD20− CD3− cells. CD4 was used to segregate the CD3+ T cells into populations of CD3+ CD4− and CD3+ CD4+ T cells.

FIG. 3 illustrates the kinetic responses of different discrete cell populations in the normal samples. The line graphs contained in FIG. 3 plot the activation levels observed in all of the donors over the time intervals at which they were measured. The different concentrations of IL-6 tabulated above are represented by solid and dashed lines. Generally, the normal samples demonstrated similar activation profiles over time according to the concentration of sample given. Different concentrations of the modulator IL-6 yielded dramatically different activation profiles for some of the Stat phosphoproteins measured. For example, IL-6-induced pStat3 response varied at early time points (5-15 minutes) for the different concentrations of IL-6 but became more uniform at later time points. This uniformity of response supports the idea that normal cells exhibit a narrow range of activation.

Different discrete cell populations demonstrated unique responses to modulation. The neutrophils exhibited very low IL-6 induced activation as compared to the CD4+ T cells and monocytes. Between the CD4+ T cells and monocytes, several differences in activation profiles were observed. Monocytes showed a peak activation of IL-6-induced pStat1 activity at a different time point than the CD4+ T cells. Although both the monocytes and the CD4+ T cells demonstrated a drop-off in pStat3 activity after 15 minutes, the drop-off was much more dramatic in the monocytes. The difference in the slopes is illustrated in FIG. 3 by the use of boxes. This observation confirms the utility of using additional metrics which describe the dynamic response such as ‘slope’ and liner equations to represent dynamic response to induced activation.

What is claimed is:

1. A method of determining the status of an individual, said method comprising:
a) contacting a first cell from a first cell population from said individual with at least a first modulator;
b) contacting a second cell from a second cell population from said individual with at least a second modulator;
c) determining an activation level of at least one activatable element in said first cell and said second cell;
d) creating a response panel for said individual comprising said determined activation levels of said activatable elements; and
e) identifying the status of said individual, wherein said identifying is based on said response panel.

2. The method of claim 1, further comprising applying a classifier to said response panel, wherein the classifier comprises a set of activation levels values, and where the classifier is used to determine whether the response panel is associated with the status of the individual.

3. The method of claim 2, wherein further comprising generating a classification value based on the response panel, wherein the classification value specifies whether the individual is associated with a status of the individual.

4. The method of claim 1, further comprising determining a causal association between said first cell and said second cell based on said response panel, wherein said causal association is indicative of a state of a cell network.

5. The method of claim 1, wherein said first and second modulator are selected from the group consisting of growth factor, mitogen, cytokine, chemokine, adhesion molecule modulator, hormone, small molecule, polynucleotide, antibody, natural compound, lactone, chemotherapeutic agent, immune modulator, carbohydrate, protease, ion, reactive oxygen species, and radiation.

6. The method of claim 1, wherein said first modulator and second modulator are the same.

7. The method according to claim 6, wherein said contacting of said first cell and said contacting of said second cell is in a same culture.

8. The method of claim 1, wherein said first modulator and second modulator are different and said contacting of said first cell and said contacting of said second cell are in separate cultures.

9. The method of claim 8, wherein said contacting of said first cell and/or said contacting of said second cell is before isolation of said first cell and/or said second cell from said individual.

10. The method of claim 1 wherein said activation level is based on the activation state selected from the group consisting of extracellular protease exposure, novel hetero-oligomer formation, glycosylation state, phosphorylation state, acetylation state, methylation state, biotinylation state, glutamylation state, glycylation state, hydroxylation state, isomerization state, prenylation state, myristoylation state, lipoylation state, phosphopantetheinylation state, sulfation state, ISGylation state, nitrosylation state, palmitoylation state, SUMOylation state, ubiquitination state, neddylation state, citrullination state, deamidation state, disulfide bond formation state, proteolytic cleavage state, translocation state, changes in protein turnover, multi-protein complex state, oxidation state, multi-lipid complex, and biochemical changes in cell membrane.

11. The method of claim 10 wherein said activation state is a phosphorylation state.

12. The method of claim 1 wherein said activatable element is selected from the group consisting of proteins, carbohydrates, lipids, nucleic acids and metabolites.

13. The method of claim 12 wherein said activatable element is a protein having a phosphoryled state and/or dephosphorylated state.

14. The method of claim 1 wherein said method further comprises determining the presence or absence of one or more cell surface markers, intracellular markers, or combination thereof in said first cell and/or said second cell.

15. The method of claim 14 wherein said cell surface markers and said intracellular markers are independently selected from the group consisting of proteins, carbohydrates, lipids, nucleic acids and metabolites.

16. The method of claim 14 wherein said determining of the presence or absence of one or more cell surface markers or intracellular markers comprises determining the presence or absence of an epitope in both activated and non-activated forms of said cell surface markers or said intracellular markers.

17. The method of claim 14 wherein the status of said individual is based on both the activation levels of said activatable elements and the presence or absence of said one or more cell surface markers, intracellular markers, or combination thereof.

18. The method of claim 1 wherein said activation level is determined by a process comprising the binding of a binding element which is specific to a particular activation state of the particular activatable element.

19. The method of claim 18, wherein said binding element comprises an antibody.

20. The method of claim 18, wherein said binding elements are distinguishably labeled.

21. The method of claim 20, wherein said distinguishably labeled binding element is directly labeled with a detectable label.

22. The method of claim 21, wherein said detectable label is selected from the group consisting of: radioisotopes, heavy isotopes, fluorescers, FRET labels, enzymes, particles, and chemiluminescers.

23. The method of claim 1, wherein the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells.

24. The method of claim 1, wherein the step of determining the activation level comprises the use of flow cytometry.

25. The method of claim 1, wherein said determining is quantitative.

26. The method of claim 1, wherein said determining is relative to a control value.

27. The method of claim 26, wherein said control value is included in said response panel.

28. The method of claim 1, further comprising comparing said response panel to a classifier.

29. The method of claim 28, wherein said classifier is used to identify the status of said individual.

30. The method of claim 1, wherein said status is the classification, diagnosis, or prognosis of a condition.

31. The method of claim 30, wherein the AUC value in the classification, diagnosis, or prognosis of said condition is higher than 0.6.

32. The method of claim 30, wherein the p value in the classification, diagnosis, or prognosis of said condition is below 0.05.

33. The method of claim 30, wherein the positive predictive value (PPV) in the classification, diagnosis, or prognosis of said condition is higher than 80%.

34. The method of claim 30, wherein the negative predictive value (NPV) in the classification, diagnosis, or prognosis of said condition is higher than 80%.

35. The method of claim 30, wherein said condition is an immunologic, inflammatory, transplant rejection, infections, vaccines state responses, malignant, or proliferative disorder or a combination thereof.

36. The method of claim 35, wherein the condition is a malignant disorder.

37. The method of claim 36, wherein the malignant disorder is a solid tumor or a hematologic malignancy.

38. The method of claim 36, wherein said malignant disorder is non-B cell lineage derived.

39. The method of claim 38, wherein said non-B cell lineage derived malignant disorder is selected from the group consisting of Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B cell atypical immune lymphoproliferations.

40. The method of claim 39, wherein said non-B cell lineage derived malignant disorder is AML.

41. The method of claim 36, wherein said malignant disorder is a B cell or B cell lineage derived disorder.

42. The method of claim 41, wherein said malignant disorder is a B-Cell or B cell lineage derived disorder selected from the group consisting of Chronic Lymphocytic Leukemia (CLL), B cell lymphocyte lineage leukemia, B cell lymphocyte lineage lymphoma, Multiple Myeloma, and plasma cell disorders.

43. The method of claim 42, wherein said B-Cell or B cell lineage derived disorder is CLL.

44. The method of claim 1, wherein the status is a predicted response to a treatment for a pre-pathological or pathological condition, or a response to treatment for a pre-pathological or pathological condition.

45. The method of claim 1, further comprising predicting a response to a treatment for a pre-pathological or pathological condition.

46. The method of claim 45, wherein the AUC value in predicting a response to a treatment is higher than 0.6.

47. The method of claim 45, wherein the p value in predicting a response to a treatment is below 0.05.

48. The method of claim 45, wherein the PPV in predicting a response to a treatment of said condition is higher than 80%.

49. The method of claim 45, wherein the negative predictive value NPV in predicting a response to a treatment is higher than 80%.

50. The method of claim 1, wherein the activation levels of a plurality of intracellular activatable elements in said first cell and/or second cell is determined.

51. The method of claim 1, further comprising determining a causal association between said first cell and said second cell.

52. A computer-implemented method of classifying activation state data derived from a population of cells according to a characteristic, the method comprising:
providing a computer comprising memory and a processor;
identifying an activation state data associated with an individual, wherein the activation state data is derived from at least two discrete populations of cells sampled from an individual;
generating a classification value, wherein said classification value specifies whether the individual is associated with a health status responsive to applying a classifier to the activation state data associated with the individual; wherein the classifier comprises a set of activation state values used to determine whether cells in different discrete populations of cells are associated with the status; and
storing the classification value in memory associated with the computer.

53. The method of claim 52, wherein the classification value represents one or more of the following: a diagnosis, a prognosis and a predicted response to treatment.

54. The method of claim 52, where the activation state data is received from a third party and further comprising:
transmitting the classification value to the third party.

55. The method of claim 52, further comprising:
identifying whether the activation state data is associated with a first discrete population of cells or a second distinct population of cells based on at least a first level of an activation state associated with an activatable element.

56. The method of claim 52, wherein identifying whether the activation state data is associated with the first discrete population of cells or the second distinct population of cells comprises gating the activation state data based on the at least a first level of an activation state associated with the activatable element.

57. The method of claim 55, wherein identifying whether the activation state data is associated with the first discrete population of cells or the second discrete population of cells comprises iteratively binning the activation state data based on at least a first level of an activation state associated with an activatable element.

58. The method of claim 57, wherein the first discrete population of cells is a rare population of cells and the first discrete population of cells is identified responsive to iteratively binning the activation state data based on at least a first level of an activation state associated with an activatable element.

59. The method of claim 52, further comprising generating the classifier based on activation state data derived from a plurality of discrete populations of cells that are known to be associated with the status and a plurality of discrete populations of cells that are know not to be associated with the status.

60. The method of claim 59, wherein the activation state data is further associated with a plurality of time points and generating the classifier further comprises:
generating a model of the data over the different time points, where the model represents communications between the heterogeneous populations of cells over the plurality of time points;
generating a series of descriptive values based on the model; and
generating the classifier based on the series of descriptive values.

61. The method of claim 59, wherein generating the classifier comprises cross-validating the classifier.
